(19) United States
(12) Patent Application Publication
(10) Pub. No.: US 2015/0225774 A1
(43) Pub. Date: Aug. 13, 2015

(54) METHODS, WORKFLOWS, KITS, APPARATUSES, AND COMPUTER PROGRAM MEDIA FOR NUCLEIC ACID SAMPLE PREPARATION FOR NUCLEIC ACID SEQUENCING

(71) Applicant: Life Technologies Corporation, Carlsbad, CA (US)

(72) Inventors: Maxim G. BREVNOV, Union City, CA (US); Elena BOLCHAKOVA, Union City, CA (US); Manohar FURTADO, Fremont, CA (US)

(21) Appl. No.: 14/603,076

(22) Filed: Jan. 22, 2015

Related U.S. Application Data
(63) Continuation of application No. 13/171,322, filed on Jun. 28, 2011, now abandoned.
(60) Provisional application No. 61/359,307, filed on Jun. 28, 2010.

(51) Int. Cl. C12Q 1/68

(52) U.S. Cl. CPC C12Q 1/6806

(57) ABSTRACT

A method for preparing a nucleic acid sample for nucleic acid sequencing includes amplifying a nucleic acid target sequence using a primer bound to a first capture substrate; capturing an amplified nucleic acid product by the first capture substrate; generating at least one sequencing ladder from the amplified nucleic acid product using at least one sequencing primer; capturing the at least one sequencing ladder by hybridizing the at least one sequencing ladder to a complementary capture compound on a second capture substrate; and removing the at least one sequencing ladder from the second capture substrate. The first and/or second capture substrate may include a magnetic particle. Other methods, workflows, kits, and computer program media for nucleic acid sample preparation are also disclosed.

This application is a continuation of U.S. Nonprovisional application Ser. No. 13/171,322, filed Jun. 28, 2011, which claims the benefit of U.S. Provisional Application No. 61/359,307, filed Jun. 28, 2010, which disclosures are incorporated herein by reference in its entirety.

### 1. FIELD

The present application relates to methods, workflows, kits, apparatuses, and computer program media for the preparation of one or more nucleic acid samples for nucleic acid sequencing.

### 2. BACKGROUND

Upon completion of the Human Genome project, one focus of the sequencing industry has shifted to finding higher throughput and/or lower cost sequencing technologies. In making sequencing higher throughput and/or less expensive, a goal is to make sequencing technology more accessible. Sequencing platforms and methods that provide sample preparation for larger numbers of samples and/or analysis in a shorter period of time may help to attain these goals.

Current sequencing workflows often require many preparation steps, many of which involve human sample manipulation. Exemplary preparation steps may include amplification of a sample, cleaning and/or quantification of one or more amplification products, and/or cleaning to remove excess reactants or other impurities after sequencing reactions to permit sequence determination of the purified sequencing reaction products.

In various conventional workflows, preparing nucleic acid samples for sequencing requires multiple manual liquid transfers. Manual liquid transfers may increase the amount of time required for the sample preparation and/or increase the probability of errors, including contamination, in the preparation of the samples. Moreover, for assays involving low volumes of sample, transferring the low volumes of liquid can pose difficulties.

The conventional sample preparation workflows also require multiple cleaning or purification steps to ensure all reactants are removed from the nucleic acid sample prior to sequencing. Other steps that may be required by conventional sample preparation workflows also include enzymatic removal steps or desalting steps. For example, exoSAP is often used to enzymatically remove leftover primers and nucleotides.

Following amplification of the nucleic acid sample, quantification is often necessary to ascertain how much amplified sample is available. For sequencing reactions, a certain amount (e.g., concentration) of the amplified nucleic acid sample may be required. Therefore, it may be necessary to determine that the amplification produced the desired amount before performing the sequencing reactions.

There is a need in the art of nucleic acid sequencing for sample preparation methods and workflows that can increase the number of nucleic acid samples sequenced, reduce the time required to prepare nucleic acid samples for sequencing, and/or reduce the probability of human error during the preparation of nucleic acid samples for sequencing. Further, it can be desirable to reduce the number of steps required to prepare nucleic acid samples for sequencing.

Embodiments of the present invention may solve one or more of the above-mentioned problems. Other features and/or advantages may become apparent from the description which follows.

### SUMMARY

According to an exemplary embodiment of the invention, there is provided a method for preparing a nucleic acid sample for nucleic acid sequencing, including amplifying a nucleic acid target sequence using a primer bound to a first capture substrate; capturing an amplified nucleic acid product by the first capture substrate; generating at least one sequencing ladder from the amplified nucleic acid product using at least one sequencing primer; capturing the at least one sequencing ladder by hybridizing the at least one sequencing ladder to a complementary capture compound on a second capture substrate; and removing the at least one sequencing ladder from the second capture substrate.

In this method, the first capture substrate may include a capture compound, and the primer may include a prey moiety configured to form a specific binding pair with the capture compound, in which case capturing the amplified nucleic acid product by the first capture substrate may include attracting the magnetic particle to a magnet. The specific binding pair may be a biotin-avidin binding pair. The sequencing primer may include a prey moiety, in which case hybridizing the sequencing ladder to a complementary capture compound on a second capture substrate may include hybridizing the prey moiety of the at least one sequencing primer to the complementary capture compound on the second capture substrate. The first capture substrate may include a magnetic particle. The second capture substrate may also include a magnetic particle. The method may further include quantifying the amplified nucleic acid product using a pre-determined quantity of the first capture substrate.

According to another exemplary embodiment of the invention, there is provided a method for preparing a nucleic acid sample for nucleic acid sequencing, including: amplifying a nucleic acid target sequence with a first primer and a second primer to generate complementary amplified nucleic acid sequences, the first primer being bound to a first capture substrate; hybridizing the complementary amplified nucleic acid sequences; removing the first capture substrate bound to the first primer from which one of the hybridized amplified nucleic acid sequences was generated from remaining amplification reaction products; generating a forward sequencing ladder and a reverse sequencing ladder with a forward sequencing primer and a reverse sequencing primer; and separating the forward sequencing ladder from the reverse sequencing ladder by hybridizing the forward sequencing ladder to a capture compound on a second capture substrate. The first capture substrate may be magnetic. The second capture substrate may also be magnetic.

According to another exemplary embodiment of the invention, there is provided a kit for nucleic acid sample preparation, including a plurality of containers; a set of nucleic acid amplification reagents including an amplification primer; a first capture substrate for capturing the amplification primer; a set of sequencing reaction reagents; and a second capture substrate for capturing a sequencing reaction product.

In this kit, the amplification primer may include a prey moiety capable of forming a specific binding pair with a capture compound on the first capture substrate. The first capture substrate may be magnetic. The second capture substrate may also be magnetic, and it may include a nucleic acid sequence capable of hybridizing to the sequencing reaction product. The sequencing reaction reagents may include a primer, and the sequencing reaction reagents may include dideoxynucleotides. The nucleic acid amplification reagents and the sequencing reaction reagents may be lyophilized.

According to another exemplary embodiment of the invention, there is provided a method for preparing a nucleic acid sample for nucleic acid sequencing, including: (1) providing a first container containing a lyophilized reagent for PCR, a magnetic particle, a forward primer, and a reverse primer; (2) loading a nucleic acid sample including target nucleic acid in the first container; and (3) allowing the nucleic acid sample to mix with the lyophilized reagent for PCR, magnetic particle, forward primer, and reverse primer in the first container.

In this method, the magnetic particle may be a bead comprising a magnetic core covered by a plastic coating, may include streptavidin on a surface thereof, and may have a diameter between about 1 μm and about 5 μm. At least one of the forward primer and the reverse primer may be attached to the magnetic particle. The forward primer may be attached to the magnetic particle while the reverse primer is not attached to the magnetic particle. Allowing the nucleic acid sample to mix may include mixing by pipetting the nucleic acid sample up and down the first container, by vibrating the first container, and/or by agitating the first container axially and/or rotationally.

The method may further include subjecting the target nucleic acid to an amplification reaction using thermal cycling to produce an amplified sample including hybridized forward and reverse amplification strands attached to the magnetic particle, and inserting a magnet into the first container to attract the magnetic particle to which are attached the hybridized forward and reverse amplification strands. The magnet may be a plastic-sheathed magnet, and it may be a magnetic rod contained substantially concentrically within a non-magnetic sheath that is independently moveable in an axial direction relative to the non-magnetic sheath. The method may further include providing a second container containing a wash solution; transferring the amplified sample comprising hybridized forward and reverse amplification strands attached to the magnetic particle from the first container to the second container with the magnet; and washing the amplified sample comprising hybridized forward and reverse amplification strands attached to the magnetic particle using the wash solution in the second container to remove unreacted nucleotides, polymerase, and/or primers that may be on the hybridized forward and reverse amplification strands. The wash solution may include about 20 mM Tris-HCl (tris(hydroxymethyl)aminomethane hydrochloric acid) and about 0.1% Tween (polyoxyethylene (20) sorbitan monolaurate).

The method may further include providing a third container containing forward and reverse sequencing primers; transferring the washed amplified sample comprising hybridized forward and reverse amplification strands attached to the magnetic particle from the second container to the third container with the magnet; and subjecting the washed amplified sample to a sequencing reaction in the third container using thermal cycling to generate forward and reverse sequencing ladders of the amplified sample. The sequencing reaction may include thermal cycling. It may further include transferring the amplified sample comprising hybridized forward and reverse amplification strands attached to the magnetic particle from the third container to the second container with the magnet; and leaving the forward and reverse sequencing ladders of the amplified sample in the third container.

The method may further include providing a fourth container containing a forward sequencing capture substrate; transferring the forward sequencing capture substrate from the fourth container to the third container with the magnet; and hybridizing the forward sequencing capture substrate with prey moieties present on the forward sequencing ladders of the amplified sample in the third container.

The method may further include providing a fifth container containing a wash solution; transferring the forward sequencing capture substrate hybridized with prey moieties present on the forward sequencing ladders from the third container to the fifth container with the magnet; and washing the forward sequencing capture substrate hybridized with prey moieties present on the forward sequencing ladders using the wash solution in the fifth container. The wash solution may include about 20 mM Tris-HCl (tris(hydroxymethyl)aminomethane hydrochloric acid) and about 0.1% Tween (polyoxyethylene (20) sorbitan monolaurate).

The method may further include providing a sixth container containing a denaturing compound; transferring the washed forward sequencing capture substrate hybridized with prey moieties present on the forward sequencing ladders from the fifth container to the sixth container with the magnet; denaturing the forward sequencing capture substrate hybridized with prey moieties present on the forward sequencing ladders in the sixth container; selectively eluting the forward sequencing ladders in the sixth container; and transferring the forward sequencing capture substrate from the sixth container to the fourth container using the magnet. The denaturing compound may include formamide.

The method may further include providing a seventh container containing a reverse sequencing capture substrate; transferring the reverse sequencing capture substrate from the seventh container to the third container with the magnet; and hybridizing the reverse sequencing capture substrate with the reverse sequencing ladders. It may further include transferring the reverse sequencing capture substrate hybridized with the reverse sequencing ladders from the third container to the fifth container with the magnet; and washing the reverse sequencing capture substrate hybridized with the reverse sequencing ladders using the wash solution in the fifth container.

The method may further include providing an eighth container containing a denaturing compound; transferring the washed reverse sequencing capture substrate hybridized with the reverse sequencing ladders from the fifth container to the eighth container with the magnet; denaturing the reverse sequencing capture substrate hybridized with the reverse sequencing ladders in the eighth container; selectively eluting the reverse sequencing ladders in the eighth container; and transferring the reverse sequencing capture substrate from the eighth container to the seventh container with the magnet. The denaturing compound may include formamide.

Finally, the method may further include subjecting the forward sequencing ladders in the sixth container and/or the reverse sequencing ladders in the eighth container to one or more further reactions and/or analysis. And it may further include subjecting the forward sequencing ladders in the sixth container and/or the reverse sequencing ladders in the eighth container to capillary electrophoresis. The steps of providing the first, second, third, fourth, fifth, sixth, seventh, and eighth containers and their contents may be performed before any of the other steps involving any reactions in the containers or transfers between the containers. These steps may also be performed simultaneously within an automated robotic system.

According to another exemplary embodiment of the invention, there is provided a method for automatically preparing nucleic acid samples for nucleic acid sequencing with high-throughput, including: (1) placing, in an apparatus configured to manipulate fluids and magnetic particles, (a) a reagent for PCR, a magnetic particle, a forward primer, and a reverse primer in a first container, (b) a wash solution in a second container, (c) forward and reverse sequencing primers in a third container, (d) a forward sequencing capture substrate in a fourth container, (e) a reverse sequencing capture substrate in a fifth container, (f) a wash solution in a sixth container, (g) a denaturing agent in a seventh container, and (h) a denaturing agent in an eighth container; (2) loading a nucleic acid sample including target nucleic acid in the first container; and (3) allowing the nucleic acid sample to mix with the reagent for PCR, magnetic particle, forward primer, and reverse primer in the first container.

In this method, the magnetic particle may be a bead including a magnetic core covered by a plastic coating, may include streptavidin on a surface thereof, and may have a diameter between about 1 μm and about 5 μm. At least one of the forward primer and the reverse primer may be attached to the magnetic particle. The forward primer may be attached to the magnetic particle while the reverse primer is not attached to the magnetic particle. Allowing the nucleic acid sample to mix may include mixing by moving a fluid dispensing device for dispensing the nucleic acid sample up and down the first container, by vibrating the first container, and/or by agitating the first container axially and/or rotationally. At least one of the denaturing agent of the seventh container and the denaturing agent of the eighth container may include formamide.

The method may further include automatically subjecting the target nucleic acid to an amplification reaction using thermal cycling to produce an amplified sample comprising hybridized forward and reverse amplification strands attached to the magnetic particle. It may further include automatically inserting a magnet into the first container to attract the magnetic particle to which are attached the hybridized forward and reverse amplification strands. The magnet may be a plastic-sheathed magnet, and it may include a magnetic rod contained substantially concentrically within a non-magnetic sheath, which magnetic rod may be independently moveable in an axial direction relative to the non-magnetic sheath.

The method may further include automatically transferring the amplified sample comprising hybridized forward and reverse amplification strands attached to the magnetic particle from the first container to the second container with the magnet; and washing the amplified sample comprising hybridized forward and reverse amplification strands attached to the magnetic particle using the wash solution in the second container to remove unreacted nucleotides, polymerase, and/or primers that may be on the hybridized forward and reverse amplification strands.

The method may further include automatically transferring the washed amplified sample comprising hybridized forward and reverse amplification strands attached to the magnetic particle from the second container to the third container with the magnet; and automatically subjecting the washed amplified sample to a sequencing reaction in the third container using thermal cycling to generate forward and reverse sequencing ladders of the amplified sample. It may further include automatically transferring the amplified sample comprising hybridized forward and reverse amplification strands attached to the magnetic particle from the third container to the second container with the magnet; and leaving the forward and reverse sequencing ladders of the amplified sample in the third container.

The method may further include automatically transferring the forward sequencing capture substrate from the fourth container to the third container with the magnet; and hybridizing the forward sequencing capture substrate with prey moieties present on the forward sequencing ladders of the amplified sample in the third container. It may further include automatically transferring the forward sequencing capture substrate hybridized with prey moieties present on the forward sequencing ladders from the third container to the sixth container with the magnet; and washing the forward sequencing capture substrate hybridized with prey moieties present on the forward sequencing ladders using the wash solution in the sixth container.

The method may further include automatically transferring the washed forward sequencing capture substrate hybridized with prey moieties present on the forward sequencing ladders from the sixth container to the seventh container with the magnet; denaturing the forward sequencing capture substrate hybridized with prey moieties present on the forward sequencing ladders in the seventh container; selectively eluting the forward sequencing ladders in the seventh container; and automatically transferring the forward sequencing capture substrate from the seventh container to the fourth container using the magnet.

The method may further include automatically transferring the reverse sequencing capture substrate from the fifth container to the third container with the magnet; and hybridizing the reverse sequencing capture substrate with the reverse sequencing ladders. It may further include automatically transferring the reverse sequencing capture substrate hybridized with the reverse sequencing ladders from the third container to the sixth container with the magnet; and washing the reverse sequencing capture substrate hybridized with the reverse sequencing ladders using the wash solution in the sixth container.

The method may further include automatically transferring the washed reverse sequencing capture substrate hybridized with the reverse sequencing ladders from the sixth container to the eighth container with the magnet; denaturing the reverse sequencing capture substrate hybridized with the reverse sequencing ladders in the eighth container; selectively eluting the reverse sequencing ladders in the eighth container; and automatically transferring the reverse sequencing capture substrate from the eighth container to the fifth container with the magnet.

Finally, the method may further include automatically subjecting the forward sequencing ladders in the seventh container and/or the reverse sequencing ladders in the eighth container to capillary electrophoresis.

According to another exemplary embodiment of the invention, there is provided a method for increasing nucleic acid sample preparation throughput, including (1) placing, in an apparatus configured to manipulate fluids, (a) a reagent for PCR, a magnetic particle, a forward primer, and a reverse primer in a first container, the forward primer being attached to the magnetic particle, and (b) a wash solution in a second container; (2) loading a nucleic acid sample including target nucleic acid in the first container; (3) mixing the nucleic acid sample with the reagent for PCR, magnetic particle, forward primer, and reverse primer in the first container by at least one of moving a pipetting device for pipetting the nucleic acid sample up and down the first container, vibrating the first container, and agitating the first container axially and/or rotationally; (4) automatically subjecting the target nucleic acid to an amplification reaction using thermal cycling to produce an amplified sample comprising hybridized forward and reverse amplification strands attached to the magnetic particle; (5) automatically inserting a magnet into the first container to attract the magnetic particle to which are attached the hybridized forward and reverse amplification strands; and (6) automatically transferring the amplified sample comprising hybridized forward and reverse amplification strands attached to the magnetic particle from the first container to the second container with the magnet.

In this method, the magnet may include a magnetic rod contained substantially concentrically within a non-magnetic sheath, which magnetic rod may be independently moveable in an axial direction relative to the non-magnetic sheath.

According to another exemplary embodiment of the invention, there is provided a method for increasing nucleic acid sample preparation throughput, including: (1) placing, in an apparatus configured to manipulate fluids, (a) a reagent for PCR, a magnetic particle, a forward primer, and a reverse primer in a first container, (b) a wash solution in a second container, (c) forward and reverse sequencing primers in a third container, and (d) a forward sequencing capture substrate in a fourth container; (2) loading a nucleic acid sample including target nucleic acid in the first container and allowing the nucleic acid sample to mix with the reagent for PCR, magnetic particle, forward primer, and reverse primer in the first container; (3) automatically subjecting the target nucleic acid to an amplification reaction using thermal cycling to produce an amplified sample comprising hybridized forward and reverse amplification strands attached to the magnetic particle; (4) automatically inserting a magnet into the first container to attract the magnetic particle to which are attached the hybridized forward and reverse amplification strands; and (5) automatically transferring the amplified sample comprising hybridized forward and reverse amplification strands attached to the magnetic particle from the first container to the second container with the magnet for washing in the second container.

In this method, the magnet may include a magnetic rod contained substantially concentrically within a non-magnetic sheath and independently moveable in an axial direction relative to the non-magnetic sheath. The method may further include automatically transferring the amplified sample comprising hybridized forward and reverse amplification strands attached to the magnetic particle from the second container to the third container with the magnet; automatically subjecting the amplified sample to a sequencing reaction in the third container using thermal cycling to generate forward and reverse sequencing ladders of the amplified sample; automatically transferring the amplified sample comprising hybridized forward and reverse amplification strands attached to the magnetic particles from the third container to the second container with the magnet, and leaving the forward and reverse sequencing ladders of the amplified sample in the third container; and automatically transferring the forward sequencing capture substrate from the fourth container to the third container with the magnet, and hybridizing the forward sequencing capture substrate with prey moieties present on the forward sequencing ladders of the amplified sample in the third container.

The method may further include placing a wash solution in a fifth container, and a denaturing agent in a sixth container. It may further include automatically transferring the forward sequencing capture substrate hybridized with prey moieties present on the forward sequencing ladders from the third container to the fifth container with the magnet, and washing the forward sequencing capture substrate hybridized with prey moieties present on the forward sequencing ladders using the wash solution in the fifth container; automatically transferring the forward sequencing capture substrate hybridized with prey moieties present on the forward sequencing ladders from the fifth container to the sixth container with the magnet; denaturing the washed forward sequencing capture substrate hybridized with prey moieties present on the forward sequencing ladders in the sixth container; selectively eluting the forward sequencing ladders in the sixth container; and automatically transferring the forward sequencing capture substrate from the sixth container to the fourth container using the magnet.

Finally, the method may further include automatically subjecting the forward sequencing ladders in the sixth container to capillary electrophoresis.

According to another exemplary embodiment of the invention, there is provided a method for increasing nucleic acid sample preparation throughput, including: (1) placing, in an apparatus configured to manipulate fluids, (a) a reagent for PCR, a magnetic particle, a forward primer, and a reverse primer in a first container, (b) a wash solution in a second container, (c) forward and reverse sequencing primers in a third container, and (d) a reverse sequencing capture substrate in a fourth container; (2) loading a nucleic acid sample including target nucleic acid in the first container and allowing the nucleic acid sample to mix with the reagent for PCR, magnetic particle, forward primer, and reverse primer in the first container; (3) automatically subjecting the target nucleic acid to an amplification reaction using thermal cycling to produce an amplified sample comprising hybridized forward and reverse amplification strands attached to the magnetic particle; (4) automatically inserting a magnet into the first container to attract the magnetic particle to which are attached the hybridized forward and reverse amplification strands; and (5) automatically transferring the amplified sample comprising hybridized forward and reverse amplification strands attached to the magnetic particle from the first container to the second container with the magnet for washing in the second container.

In this method, the magnet may include a magnetic rod contained substantially concentrically within a non-magnetic sheath and independently moveable in an axial direction relative to the non-magnetic sheath. The method may further include automatically transferring the amplified sample comprising hybridized forward and reverse amplification strands attached to the magnetic particle from the second container to the third container with the magnet; automatically subjecting the amplified sample to a sequencing reaction in the third container using thermal cycling to generate forward and reverse sequencing ladders of the amplified sample; automatically transferring the amplified sample comprising hybridized forward and reverse amplification strands attached to the magnetic particle from the third container to the second container with the magnet, and leaving the forward and reverse sequencing ladders of the amplified sample in the third container; and automatically transferring the reverse sequencing capture substrate from the fourth container to the third container with the magnet, and hybridizing the reverse sequencing capture substrate with the reverse sequencing ladders of the amplified sample in the third container.

The method may further include placing a wash solution in a fifth container, and a denaturing agent in a sixth container. It may further include automatically transferring the reverse sequencing capture substrate hybridized with the reverse sequencing ladders from the third container to the fifth container with the magnet, and washing the reverse sequencing capture substrate hybridized with the reverse sequencing ladders using the wash solution in the fifth container; automatically transferring the washed reverse sequencing capture substrate hybridized with the reverse sequencing ladders from the fifth container to the sixth container with the magnet; denaturing the reverse sequencing capture substrate hybridized with the reverse sequencing ladders in the sixth container; selectively eluting the reverse sequencing ladders in the sixth container; and automatically transferring the reverse sequencing capture substrate from the sixth container to the fourth container using the magnet.

Finally, the method may further include automatically subjecting the reverse sequencing ladders in the sixth container to capillary electrophoresis.

According to another exemplary embodiment of the invention, there is provided a kit for nucleic acid sample preparation, including a plurality of containers; a lyophilized reagent for PCR; a magnetic particle; a forward primer; and a reverse primer.

In this kit, the magnetic particle may include streptavidin on a surface thereof, and may have a diameter between about 1 μm and about 5 μm. The forward primer may be attached to the magnetic particle while the reverse primer is not attached to the magnetic particle. Alternatively, the reverse primer may be attached to the magnetic particle while the forward primer is not be attached to the magnetic particle. The wash solution may include about 20 mM Tris-HCl (tris(hydroxymethyl)aminomethane hydrochloric acid) and about 0.1% Tween (polyoxyethylene (20) sorbitan monolaurate). The kit may further include forward and reverse sequencing primers, and it may further include forward and reverse sequencing capture substrates. The kit may further include a denaturing compound including formamide.

According to another exemplary embodiment of the invention, there is provided an apparatus for nucleic acid sample preparation, including a plurality of containers; at least two thermal cycling elements configured to subject at least two of the containers to thermal cycling; and a magnet comprising a magnetic rod contained substantially concentrically within a non-magnetic sheath and independently moveable in an axial direction relative to the non-magnetic sheath, the magnet being controlled to be insertable into any one of the containers and moveable from any one of the containers to any other one of the containers.

According to another exemplary embodiment of the invention, there is provided a computer readable medium including computer readable instructions, which, when executed by a computer in or in communication with a fluid-handling apparatus including a plurality of containers and a magnet, control the apparatus to (1) place (a) a reagent for PCR, a magnetic particle, a forward primer, and a reverse primer in a first container, at least one of the forward primer and the reverse primer being attached to the magnetic particle, (b) a wash solution in a second container, (c) forward and reverse sequencing primers in a third container, (d) a forward sequencing capture substrate in a fourth container, (e) a reverse sequencing capture substrate in a fifth container, (f) a wash solution in a sixth container, (g) a denaturing agent in a seventh container, and (h) a denaturing agent in an eighth container; (2) load a nucleic acid sample including target nucleic acid in the first container; and (3) allow the nucleic acid sample to mix with the reagent for PCR, magnetic particle, forward primer, and reverse primer in the first container.

The instructions may further control the apparatus to mix the contents of the first container by moving a fluid dispensing device for dispensing the nucleic acid sample up and down the first container, by vibrating the first container, and/or by agitating the first container axially and/or rotationally. They may further control the apparatus to subject the target nucleic acid to an amplification reaction using thermal cycling to produce an amplified sample comprising hybridized forward and reverse amplification strands attached to the magnetic particle, and to insert the magnet into the first container to attract the magnetic particle to which are attached the hybridized forward and reverse amplification strands. The magnet may include a magnetic rod contained substantially concentrically within a non-magnetic sheath, which magnetic rod may be independently moveable in an axial direction relative to the non-magnetic sheath.

The instructions may further control the apparatus to transfer the amplified sample comprising hybridized forward and reverse amplification strands attached to the magnetic particle from the first container to the second container with the magnet; and wash the amplified sample comprising hybridized forward and reverse amplification strands attached to the magnetic particle using the wash solution in the second container to remove unreacted nucleotides, polymerase, and/or primers that may be on the hybridized forward and reverse amplification. They may further control the apparatus to transfer the washed amplified sample comprising hybridized forward and reverse amplification strands attached to the magnetic particle from the second container to the third container with the magnet; and subject the washed amplified sample to a sequencing reaction in the third container using thermal cycling to generate forward and reverse sequencing ladders of the amplified sample.

The instructions may further control the apparatus to transfer the amplified sample comprising hybridized forward and reverse amplification strands attached to the magnetic particle from the third container to the second container with the magnet; and leave the forward and reverse sequencing ladders of the amplified sample in the third container. They may further control the apparatus to transfer the forward sequencing capture substrate from the fourth container to the third container with the magnet; and hybridize the forward sequencing capture substrate with prey moieties present on the forward sequencing ladders of the amplified sample in the third container. They may further control the apparatus to transfer the forward sequencing capture substrate hybridized with prey moieties present on the forward sequencing ladders from the third container to the sixth container with the magnet; and wash the forward sequencing capture substrate hybridized with prey moieties present on the forward sequencing ladders using the wash solution in the sixth container.

The instructions may further control the apparatus to transfer the washed forward sequencing capture substrate hybridized with prey moieties present on the forward sequencing ladders from the sixth container to the seventh container with the magnet; denature the forward sequencing capture substrate hybridized with prey moieties present on the forward sequencing ladders in the seventh container; selectively elute the forward sequencing ladders in the seventh container; and transfer the forward sequencing capture substrate from the seventh container to the fourth container using the magnet.

The instructions may further control the apparatus to transfer the reverse sequencing capture substrate from the fifth container to the third container with the magnet; and hybridize the reverse sequencing capture substrate with the reverse sequencing ladders. They may further control the apparatus to transfer the reverse sequencing capture substrate hybridized with the reverse sequencing ladders from the third container to the sixth container with the magnet; and wash the reverse sequencing capture substrate hybridized with the reverse sequencing ladders using the wash solution in the sixth container.

The instructions may further control the apparatus to transfer the washed reverse sequencing capture substrate hybridized with the reverse sequencing ladders from the sixth container to the eighth container with the magnet; denature the reverse sequencing capture substrate hybridized with the reverse sequencing ladders in the eighth container; selectively elute the reverse sequencing ladders in the eighth container; and transfer the reverse sequencing capture substrate from the eighth container to the fifth container with the magnet.

Finally, the instructions may further control the apparatus to subject the forward sequencing ladders in the seventh container and/or the reverse sequencing ladders in the eighth container to capillary electrophoresis.

According to another exemplary embodiment of the invention, there is provided a computer readable medium including computer readable instructions, which, when executed by a computer in or in communication with a fluid-handling apparatus including a plurality of containers and a magnet, control the apparatus to: (1) place a reagent for PCR, a magnetic particle, a forward primer, and a reverse primer in a first container, at least one of the forward primer and the reverse primer being attached to the magnetic particle; (2) place a wash solution in a second container; (3) load a nucleic acid sample including target nucleic acid in the first container and allow the nucleic acid sample to mix with the reagent for PCR, magnetic particle, forward primer, and reverse primer in the first container; (4) subject the target nucleic acid to an amplification reaction using thermal cycling to produce an amplified sample comprising hybridized forward and reverse amplification strands attached to the magnetic particle; (5) insert a magnet into the first container to attract the magnetic particle to which are attached the hybridized forward and reverse amplification strands; and (6) transfer the amplified sample comprising hybridized forward and reverse amplification strands attached to the magnetic particle from the first container to the second container with the magnet for washing in the second container.

According to another exemplary embodiment of the invention, there is provided a computer readable medium including computer readable instructions, which, when executed by a computer in or in communication with a fluid-handling apparatus including a plurality of containers and a magnet, control the apparatus to: (1) place (a) a reagent for PCR, a magnetic particle, a forward primer, and a reverse primer in a first container, at least one of the forward primer and the reverse primer being attached to the magnetic particle, (b) a wash solution in a second container, (c) forward and reverse sequencing primers in a third container, and (d) a forward sequencing capture substrate in a fourth container; (2) load a nucleic acid sample including target nucleic acid in the first container and allow the nucleic acid sample to mix with the reagent for PCR, magnetic particle, forward primer, and reverse primer in the first container; (3) subject the target nucleic acid to an amplification reaction using thermal cycling to produce an amplified sample comprising hybridized forward and reverse amplification strands attached to the magnetic particle; (4) insert a magnet into the first container to attract the magnetic particle to which are attached the hybridized forward and reverse amplification strands; and (5) transfer the amplified sample comprising hybridized forward and reverse amplification strands attached to the magnetic particle from the first container to the second container with the magnet for washing in the second container.

The instructions may further control the apparatus to transfer the amplified sample comprising hybridized forward and reverse amplification strands attached to the magnetic particle from the second container to the third container with the magnet; subject the amplified sample to a sequencing reaction in the third container using thermal cycling to generate forward and reverse sequencing ladders of the amplified sample; transfer the amplified sample comprising hybridized forward and reverse amplification strands attached to the magnetic particles from the third container to the second container with the magnet, and leave the forward and reverse sequencing ladders of the amplified sample in the third container; and transfer the forward sequencing capture substrate from the fourth container to the third container with the magnet, and hybridize the forward sequencing capture substrate with prey moieties present on the forward sequencing ladders of the amplified sample in the third container.

The instructions may further control the apparatus to place a wash solution in a fifth container and a denaturing agent in a sixth container. They may further control the apparatus to transfer the forward sequencing capture substrate hybridized with prey moieties present on the forward sequencing ladders from the third container to the fifth container with the magnet; wash the forward sequencing capture substrate hybridized with prey moieties present on the forward sequencing ladders using the wash solution in the fifth container; transfer the washed forward sequencing capture substrate hybridized with prey moieties present on the forward sequencing ladders from the fifth container to the sixth container with the magnet; denature the forward sequencing capture substrate hybridized with prey moieties present on the forward sequencing ladders in the sixth container; selectively elute the forward sequencing ladders in the sixth container; and transfer the forward sequencing capture substrate from the sixth container to the fourth container using the magnet.

Finally, the instructions may further control the apparatus to subject the forward sequencing ladders in the sixth container to capillary electrophoresis.

According to another exemplary embodiment of the invention, there is provided a computer readable medium including computer readable instructions, which, when executed by a computer in or in communication with a fluid-handling apparatus including a plurality of containers and a magnet, control the apparatus to: (1) place (a) a reagent for PCR, a magnetic particle, a forward primer, and a reverse primer in a first container, at least one of the forward primer and the reverse primer being attached to the magnetic particle, (b) a wash solution in a second container, (c) forward and reverse sequencing primers in a third container, and (d) a reverse sequencing capture substrate in a fourth container; (2) load a nucleic acid sample including target nucleic acid in the first container and allow the nucleic acid sample to mix with the reagent for PCR, magnetic particle, forward primer, and reverse primer in the first container; (3) subject the target nucleic acid to an amplification reaction using thermal cycling to produce an amplified sample comprising hybridized forward and reverse amplification strands attached to the magnetic particle; (4) insert a magnet into the first container to attract the magnetic particle to which are attached the hybridized forward and reverse amplification strands; and (5) transfer the amplified sample comprising hybridized forward and reverse amplification strands attached to the magnetic particle from the first container to the second container with the magnet for washing in the second container.

The instructions may further control the apparatus to transfer the amplified sample comprising hybridized forward and reverse amplification strands attached to the magnetic particle from the second container to the third container with the magnet; subject the amplified sample to a sequencing reaction in the third container using thermal cycling to generate forward and reverse sequencing ladders of the amplified sample; transfer the amplified sample comprising hybridized forward and reverse amplification strands attached to the magnetic particle from the third container to the second container with the magnet, and leave the forward and reverse sequencing ladders of the amplified sample in the third container; and transfer the reverse sequencing capture substrate from the fourth container to the third container with the magnet, and hybridize the reverse sequencing capture substrate with the reverse sequencing ladders of the amplified sample in the third container.

The instructions may further control the apparatus to place a wash solution in a fifth container and a denaturing agent in a sixth container. They may further control the apparatus to transfer the reverse sequencing capture substrate hybridized with the reverse sequencing ladders from the third container to the fifth container with the magnet; wash the reverse sequencing capture substrate hybridized with the reverse sequencing ladders using the wash solution in the fifth container; transfer the washed reverse sequencing capture substrate hybridized with the reverse sequencing ladders from the fifth container to the sixth container with the magnet; denature the reverse sequencing capture substrate hybridized with the reverse sequencing ladders; selectively elute the reverse sequencing ladders in the sixth container; and transfer the reverse sequencing capture substrate from the sixth container to the fourth container using the magnet.

Finally, the instructions may further control the apparatus to subject the reverse sequencing ladders in the sixth container to capillary electrophoresis.

According to another exemplary embodiment of the invention, there is provided a method for preparing a nucleic acid sample for nucleic acid sequencing, including: amplifying a nucleic acid target sequence using a first primer bound to a first capture substrate in an amplification reaction, the first capture substrate including a first magnetic particle; capturing a first amplification strand by the first capture substrate; generating at least one sequencing ladder from the first amplification strand using at least one sequencing primer in a sequencing reaction; capturing the at least one sequencing ladder, including the step of hybridizing the at least one sequencing ladder to a complementary capture compound on a second capture substrate, the second capture substrate including a second magnetic particle; and removing the at least one sequencing ladder from the second capture substrate.

In this method, the first magnetic particle may include a capture compound and the first primer may include a prey moiety configured to form a specific binding pair with the capture compound. The specific binding pair may be a biotin-avidin binding pair. The first magnetic particle may be a bead including a magnetic core covered by a plastic coating, and having a diameter between about 1 μm and about 5 μm. The first magnetic particle may include streptavidin on a surface thereof.

In this method, the at least one sequencing primer may include a prey moiety, and hybridizing the at least one sequencing ladder to a complementary capture compound on a second capture substrate may include hybridizing the prey moiety of the at least one sequencing primer to the complementary capture compound on the second capture substrate.

In this method, the magnet may include a magnetic rod contained substantially concentrically within a non-magnetic sheath. The magnetic rod may be independently moveable in an axial direction relative to the non-magnetic sheath.

In this method, the second magnetic particle may be a bead comprising a magnetic core covered by a plastic coating, having a diameter between about 1 μm and about 5 μm.

The method may further include, in the step of capturing the first amplification strand by the first capture substrate, a step of attracting the first magnetic particle to a magnet. It may further include attracting the first magnetic particle to a magnet by inserting a magnet into the amplification reaction to attract the first magnetic particle to which is attached the first amplification strand.

The method may further include quantifying the first amplification strand using a pre-determined quantity of the first capture substrate.

The method may further include providing, in the amplification reaction, a second primer configured to generate a second amplification strand capable of hybridizing with the first amplification strand. It may further include, in the step of capturing the first amplification strand by the first capture substrate, a step including: attracting the first magnetic particle to a magnet. It may further include attracting the first magnetic particle to a magnet by inserting a magnet into the amplification reaction to attract the first magnetic particle to which is attached the hybridized first and second amplification strands.

The method may further include, in the step of capturing a first amplification strand by the first capture substrate, a step including: washing an amplified sample including the first amplification strand attached to the first magnetic particle to remove unreacted nucleotides, polymerase, and/or primers that may be present.

The method may alternatively provide, in the step of capturing a first amplification strand by the first capture substrate, a step including: washing an amplified sample including the hybridized first and second amplification strands attached to the first magnetic particle to remove unreacted nucleotides, polymerase, and/or primers that may be present on the hybridized first and second amplification strands.

The method may further include a thermal cycling reaction in the sequencing reaction.

The method may include, in the step of capturing the at least one sequencing ladder, a step including: transferring the first amplification strand attached to the first magnetic particle away from the sequencing reaction, leaving the at least one sequencing ladder in the sequencing reaction. The method may alternatively include, in the step of capturing the at least one sequencing ladder, a step including: transferring the hybridized first and the second amplification strands attached to the first magnetic particle away from the sequencing reaction, leaving the at least one sequencing ladder in the sequencing reaction.

The method may include, in the step of capturing the at least one sequencing ladder, a step including: transferring the at least one sequencing ladder hybridized to the complementary capture compound on the second capture substrate away from the sequencing reaction with the magnet; and washing the at least one sequencing ladder hybridized to the complementary capture compound on the second capture substrate to remove unreacted sequencing reagents that may be present on the at least one sequencing ladder. The method may further include, in the step of capturing the at least one sequencing ladder, a step including: denaturing the at least one sequencing ladder hybridized to the complementary capture compound on the second capture substrate; and selectively eluting the at least one sequencing ladder.

The method may include subjecting the at least one sequencing ladder, after it has been freed from the second capture substrate, to capillary electrophoresis.

Finally, the method may include implementing any of the steps of the method by executing a computer readable program code using a computer or microprocessor wherein the computer or microprocessor is in or in communication with a fluid handling apparatus.

Additional objects and embodiments of the invention may be set forth in or flow from the following description, and may in part be obvious from the description, or may be learned by practice of the invention. The objects of the invention will be realized and attained by means of the elements and combinations particularly pointed out in the appended claims.

It is to be understood that both the foregoing general description and the following detailed description are exemplary and explanatory only and are not in any way restrictive of the present invention.

### BRIEF DESCRIPTION OF THE DRAWINGS

FIG. 1 is a schematic depiction of a workflow for preparing a nucleic acid sample for sequencing according to an exemplary embodiment of the present invention;

FIGS. 2A-2Q are schematic depictions of various exemplary steps of a nucleic acid sample preparation workflow according to an exemplary embodiment of the present invention;

FIG. 3 is a schematic depiction of an 8-tube strip according to an exemplary embodiment of the present invention;

FIG. 4 is a schematic depiction of a 96-well plate according to an exemplary embodiment of the present invention;

FIGS. 5A-5B are schematic depictions of an automated processor according to an exemplary embodiment of the present invention; and

FIGS. 6A-6F are schematic depictions of a sheathed magnetic rod used to transfer magnetic particles from one container to another in accordance with an exemplary embodiment of the present invention.

FIG. 7 is a representation of a gel separation depicting the comparison of a crude PCR product resulting from the amplification of an E. coli DNA sample, the unbound PCR product left behind in the amplification reaction mixture, used wash solution after washing of the magnetic particles with PCR product bound, and purified PCR product after transfer and washing, according to the workflow of FIGS. 2A-Q.

FIG. 8 is a representation of an electropherogram of the sequencing ladder obtained from amplification and subsequent forward sequencing reaction of a sample E. coli DNA according to the workflow demonstrated in FIGS. 2A-Q, where only a forward sequencing primer is present in the sequencing reaction.

FIG. 9 is a representation of an electropherogram of the sequencing ladder obtained from amplification and reverse sequencing reaction of a sample E. coli DNA according to the workflow demonstrated in FIGS. 2A-Q, where only a reverse sequencing primer is present in the sequencing reaction.

FIG. 10 is a representation of an electropherogram of the sequencing ladder obtained from amplification and subsequent forward sequencing reaction of a sample E. coli DNA, where both forward and reverse sequencing primers are present in the sequencing reaction, according to the workflow demonstrated in FIGS. 2A-Q.

FIG. 11 is a representation of an electropherogram of the sequencing ladder obtained from amplification and subsequent forward sequencing reaction of a sample E. coli DNA, where both forward and reverse sequencing primers are present in the sequencing reaction, according to the workflow demonstrated in FIGS. 2A-Q.

It is to be understood that the figures are not drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DESCRIPTION OF VARIOUS EXEMPLARY EMBODIMENTS

The section headings used herein are for organizational purposes only and are not to be construed as limiting the described subject matter in any way. When definitions of terms in incorporated references appear to differ from the definitions provided in the present teachings, the definition provided in the present teachings shall control. It will be appreciated that there is an implied “about” prior to the temperatures, concentrations, times, and other values discussed in the present description, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “have”, “having”, “include”, “includes”, and “including” are not intended to be limiting.

Unless otherwise defined, scientific and technical terms used in connection with the present teachings described herein shall have the meanings that are commonly understood by those of ordinary skill in the art. Generally, nomenclatures utilized in connection with, and techniques of, cell and tissue culture, molecular biology, and protein and oligo- or polynucleotide chemistry and hybridization described herein are those well known and commonly used in the art. Standard techniques may be used, for example, for nucleic acid purification and preparation, chemical analysis, recombinant nucleic acid, and oligonucleotide synthesis. Enzymatic reactions and purification techniques may be performed according to manufacturer's specifications or as commonly accomplished in the art or as described herein. The techniques and procedures described herein may be generally performed according to conventional methods well known in the art and as described in various general and more specific references that are cited and discussed throughout the instant specification. See, e.g., Analytical Techniques in DNA Sequencing (Edited by Brian K. Nunnaly, Taylor & Francis Group, Boca Raton, Fla., 2005); Sambrook et al., Molecular Cloning: A Laboratory Manual (Third ed., Cold Spring Harbor Laboratory Press, Cold Spring Harbor, N.Y., 2000).

As utilized in accordance with exemplary embodiments provided herein, the following terms, unless otherwise indicated, shall be understood to have the following meanings:

The terms “polynucleotide” and “oligonucleotide” as used herein are used interchangeably to refer to a polymer including natural (e.g., A, G, C, T, U) or synthetic nucleobases, or a combination of both. The backbone of the polynucleotide can be composed entirely of “native” phosphodiester linkages, or it may contain one or more modified linkages, such as one or more phosphorothioate, phosphorodithioate, phosphoramidate or other modified linkages. As a specific example, a polynucleotide may be a peptide nucleic acid (PNA), which contains amide interlinkages. Another example is L-DNA. Additional examples of synthetic bases and backbones that can be used in conjunction with the invention, as well as methods for their synthesis can be found, for example, in U.S. Pat. No. 6,001,983; Uhlman & Peyman, 1990, Chemical Review 90(4):544-584; Goodchild, 1990, Bioconjugate Chem. 1(3):165-186; Egholm et al., 1992, J. Am. Chem. Soc. 114:1895-1897; Gryaznov et al., J. Am. Chem. Soc. 116:3143-3144. Common synthetic nucleobases of which polynucleotides may be composed include 3-methlyuracil, 5,6-dihydrouracil, 4-thiouracil, 5-bromouracil, 5-thorouracil, 5-iodouracil, 6-dimethyl aminopurine, 6-methyl aminopurine, 2-aminopurine, 2,6-diamino purine, 6-amino-8-bromopurine, inosine, 5-methylcytosine, 7-deazaadenine, and 7-deazaguanosine. Additional non-limiting examples of synthetic nucleobases of which the target nucleic acid may be composed can be found in Fasman, CRC PRACTICAL HANDBOOK OF BIOCHEMISTRY AND MOLECULAR BIOLOGY, 1985, pp. 385-392; Beilstein's Handbuch der Organischen Chemie, Springer Verlag, Berlin and Chemical Abstracts, all of which provide references to publications describing the structures, properties and preparation of such nucleobases.

The terms “amplification reaction,” “amplification,” “extension reaction,” “extension” and permutations thereof, as used herein, refer to a broad range of techniques for the amplification or extension of specific polynucleotide sequences. Suitable methods of performing polynucleotide extension reactions include those described, for example, in Sambrook et al., 2001, Molecular Cloning, A Laboratory Manual, Third Edition, Cold Spring Harbor Press, N.Y., and in Ausubel et al., 1989, Current Protocols in Molecular Biology, Green Publishing Associates and Wiley Interscience, N.Y. Suitable polynucleotide extension reactions include primer extension reactions, the polymerase chain reaction, ligase chain reactions, nucleic acid sequence-based amplification and other polynucleotide extension reactions known to one of skill in the art.

The terms “sequencing ladder” or “sequence ladder” as used herein refer to a set of polynucleotides that is produced from a sequencing reaction, either a chain termination sequencing reaction, e.g., dideoxy sequencing, or from chemical cleavage sequencing, e.g., Maxam and Gilbert sequencing. The process of producing a sequencing ladder is referred to herein as “sequencing ladder generation” or “generating a sequence ladder.” Methods for generating polynucleotide sequencing ladders are well known to persons of ordinary skill in the art. Examples of methods of generating sequencing ladders can be found, among other places, in Sambrook et al, Molecular Cloning Methods: A Laboratory Manual Coldspring Harbor, Coldspring Harbor Press (1989). The different polynucleotides, i.e., members, of a specific sequencing ladder, differ in length from one another, but all members of the same ladder comprise the same oligonucleotide primer from which that sequencing ladder is derived. Thus, generating sequencing ladders from a first capturable primer and a second capturable primer that anneal to the same template priming site, but differ with respect to the identity of the sequence of the prey moiety, are considered to result in the synthesis of two different sequencing ladders. In addition to being derived from the same primer, the members of a given polynucleotide sequencing ladder are also derived from the same sequencing template. In labeled primer sequencing, four different sequencing ladders, each using a different dideoxy terminating base may be generated separately (and may subsequently be combined prior to analysis), even though only a single completed sequence may be obtained from combining the information in the four constituent sequencing ladders. When the same capturable sequencing primer and template are used to generate sequencing ladders in separate reaction vessels, the sequencing ladders produced are considered to be different sequencing ladders.

The term “family of primer extension products” as used herein refers to one or more polynucleotides generated from the same capturable primer or from different capturable primers that can be selectively immobilized by the same capture compound. For example, a family of primer extension products can be a ladder of primer extension products generated from a capturable sequencing primer, e.g., a sequencing ladder. The polynucleotides of a family of primer extension products can be substantially identical, or they can be different. For instance, a family of primer extension products generated in a sequencing reaction can have a plurality of lengths.

The term “capturable primer” as used herein refers to a molecule comprising a priming moiety and prey moiety.

The term “priming moiety” or “primer” as used herein refers to an oligonucleotide that can be used to generate a primer extension product from a template polynucleotide according to techniques known to those of skill in the art. Generally, a priming moiety hybridizes to a template polynucleotide and a primer extension product is generated enzymatically from the priming moiety. The priming moiety is incorporated into the primer extension product. For example, a priming moiety can be used to generate a polynucleotide sequencing ladder or a polynucleotide amplification product.

The term “prey moiety” refers to a compound or a portion of a compound that, together with a capture compound, forms a specific binding pair of molecules. Examples of prey moieties include polynucleotides that are capable of hybridizing with polynucleotide capture compounds. In one embodiment, the prey moiety can be a polynucleotide comprising synthetic bases, such as for example those described in U.S. Pat. No. 6,001,983, incorporated by reference in its entirety herein, capable of hybridizing to synthetic bases of a corresponding capture compound but are not capable of hybridizing to naturally occurring bases. Typically, a polynucleotide prey moiety is a polynucleotide of 5 to 35 nucleotides, for example, a polynucleotide of 15 to 20 nucleotides.

The term “specific binding pair” refers to a pair of molecules that specifically bind to one another. Binding between members of a specific binding pair is usually non-covalent. Examples of specific binding pairs include, but are not limited to antibody-antigen (or hapten) pairs, ligand-receptor pairs, biotin-avidin pairs, polynucleotides with complementary base pairs, and the like. Examples of binding pairs include pairs of complementary polynucleotides. Each specific binding pair comprises two members; however, it may be possible to find additional compounds that may specifically bind to either member of a given specific binding pair.

The term “capture compound” as used herein refers to a compound or a portion of a compound that, together with a prey moiety, forms a specific binding pair. A capture compound can selectively bind a capturable primer and thus can also selectively bind a primer extension product generated from the capturable primer. Examples of capture compounds include polynucleotides that are capable of hybridizing with polynucleotide prey moieties. Thus, a polynucleotide capture compound can be a polynucleotide that is wholly or partially complementary to a prey moiety. In some embodiments, a polynucleotide capture compound is wholly complementary to the prey moiety. A polynucleotide capture compound is typically of 5 to 35 nucleotides, for example, 15 to 20 nucleotides. In some embodiments, a polynucleotide capture compound is of the same length as a corresponding prey moiety. The capture compound can comprise synthetic bases, such as those described in U.S. Pat. No. 6,001,983, incorporated by reference herein, that are capable of hybridizing to synthetic bases of a corresponding prey moiety but not capable of hybridizing to naturally occurring bases.

The term “capture substrate” as used herein refers to a solid support having immobilized thereon one or more capture compounds. A capture substrate can be used, for example, to capture one or more families of primer extension products from a mixture or to capture one or more sequencing reaction products from a mixture. The term “capturing” as used herein in the context of capturing a substance, such as a compound or molecule, for example, with a capture substrate or a magnet, for example, refers to achieving a bound between the substance and the capture substrate or magnet sufficient so movement imparted by or upon the capture substrate or magnet will also result in some movement of the bound substance.

The term “specific elution compound” as used herein refers to a compound or a portion of a compound that can be used to selectively disrupt the binding of a specific binding pair of a prey moiety and a capture compound. The specific elution compound can, for instance, selectively bind the capture compound, or it can selectively bind the prey moiety. Examples of specific elution compounds include polynucleotides that are capable of hybridizing with polynucleotide capture compounds or with polynucleotides prey moieties. If a corresponding polynucleotide capture compound comprises synthetic bases, such as those described in U.S. Pat. No. 6,001,983, incorporated by reference herein, that are only capable of hybridizing to other synthetic bases, then the specific elution compound can also comprise such synthetic bases at appropriate positions so that the specific elution compound is capable of hybridizing to the capture compound.

The term “selective elution” as used herein refers to the selective disruption of the interaction between a family of primer extension products and a capture substrate such that the family of primer extension products can be isolated from the capture substrate. Typically, a family of primer extension products can be isolated substantially free of other captured families of primer extension products.

The term “melting temperature” or “Tₘ” refers to a quantitative expression of the stability of a hybrid of oligonucleotides. Tₘ can be calculated according to methods known to those of skill in the art. Tₘ is typically the temperature at which 50% of a given oligonucleotide is hybridized to a corresponding oligonucleotide under given conditions.

The present teachings relate to various exemplary embodiments of nucleic acid sample preparation for nucleic acid sequencing. For example, the present teachings contemplate methods of preparing nucleic acids and workflows for nucleic acid sequencing. Various exemplary embodiments of the present teachings relate to kits for use in nucleic acid sample preparation for nucleic acid sequencing.

In the various examples and embodiments described herein, the methods and workflows are described with regard to sequencing, such as, for example, dideoxynucleotide sequencing (e.g., as employed in MicroSEQ™ identification developed by Applied Biosystems (now Life Technologies Corporation)). However, as one skilled in the art would readily appreciate, the preparation methods and workflows described herein can be applied to other sequencing systems or detection techniques. The principles of nucleic acid sample preparation and workflows using the disclosed steps and procedures can be applied to other systems and methods without departing from the scope of the present teachings and claims herein.

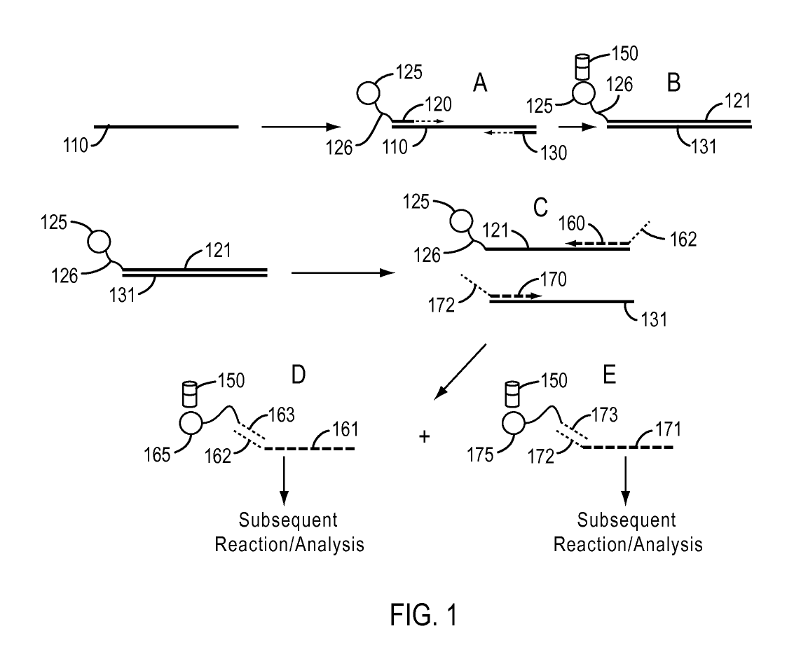

An exemplary workflow for preparing a nucleic acid sample for sequencing is shown in FIG. 1. To simplify the drawings, nucleic acid sequence strands are depicted as a line, rather than as individually connected nucleotides. Also for simplification, FIG. 1 depicts the amplification of a single target nucleic acid using a single capture substrate, capture compound, forward primer moiety and reverse primer moiety. However, those ordinarily skilled in the art would understand that the workflow can include amplification of a plurality of the same target strands using a plurality of capture substrates, capture compounds, forward primers, and reverse primers. The same applies to the generation of the sequencing ladders.

As depicted in FIG. 1, at step A, a nucleic acid sample or target strand 110 is amplified in an amplification reaction using forward and reverse primer moieties 120 and 130, respectively.

In at least one embodiment, primer moiety 120 can be a capturable primer comprising a primer moiety and a biotinylated prey moiety. Capture substrate 125 can comprise one or more capture compounds 126. In an exemplary embodiment, the one or more capture compounds 126 may comprise avidin, which forms a strong non-covalent bond with biotin. In other embodiments, the capture compound 126 and forward capturable primer 120 can comprise specific binding pairs other than biotin-avidin, such as, for example, complementary polynucleotides. In various exemplary embodiments, capture substrates can comprise particles (e.g., beads), such as, for example, magnetic particles of a type with which those of ordinary skill in the art have familiarity. The particles may comprise, for example, a magnetic core covered by a plastic coating, such as polystyrene. The particles may also comprise an active group on the surface thereof, such as, for example, streptavidin. In at least one embodiment, the particles may have a dimension (e.g., diameter) ranging from about 1 μm to about 5 μm. According to at least one embodiment, the particles may comprise Dynal® magnetic beads (available from Life Technologies Corp.) which are 2.8 μm coated beads comprising a magnetic core.

The nucleic acid target 110 may be amplified, for example, using polymerase chain reaction (PCR), by extending forward capturable primer 120. The extension of forward capturable primer 120 yields a family of primer extension products comprising the nucleic acid sequence strand 121, which is complementary to the original target sequence 110. Similarly, the reverse primer moiety 130 can be used to generate a family of primer extension products comprising the nucleic acid sequence strand 131, which is a copy of target 110 and thus complementary to strand 121. Strands 121 and 131 can then be hybridized with one another, as shown in step B of FIG. 1.

Hybridized strands 121 and 131 can be separated from the amplification reactants (e.g., unused nucleotides and polymerases) by removing capture substrate 125 to which the strand 121 is attached by virtue of capture compound 126. In accordance with at least one embodiment of the present teachings, capture substrate 125 can be magnetic and a magnet 150 may be used to attract capture substrate 125, along with the bound strand 121 and strand 131, which is hybridized to strand 121. In various exemplary embodiments, the capture substrate 125 can be a magnetic particle, bead, or the like. In at least one embodiment, the substrate can comprise a coated magnetic particle, bead, or the like, such as, for example, a magnetic particle having a polystyrene coating.

When the capture substrate 125 is magnetic, separation can occur using a magnet. The magnet may be contained within a non-magnetic sheath to keep the capture substrate 125 apart from the magnet and to provide for easy removal by removing the magnet from the sheath.

In embodiments of the present teachings wherein the specific binding pair of the capture compound and prey moiety of the forward priming moiety comprise complementary nucleotide sequences, separation of the capture compound and prey moiety may result from denaturing the hybridized sequences by heating to the melting temperature, or by using an appropriate elution compound, such as, for example, formamide.

Using capture substrates having capture compounds that form a specific binding pair to bind and to remove amplified hybridized strands (e.g., 121 and 131 in FIG. 1) can enable the desired number of amplified hybridized strands to be captured without having to rely on a separate quantification step. For example, by using a known quantity of capture substrates, the desired number of amplified strands can be removed from the amplification products by capturing the strands resulting from the extension of the capturable primers. Therefore, the number of capture substrates provided during the amplification reaction serves as a mechanism by which to verify that a desired amount of amplification products can be removed by the capture substrates after the amplification reaction. In at least one embodiment, amplification can be allowed to proceed one or more additional cycles beyond the estimated number of cycles that results in the defined amount of amplification products that can be removed. This can be done to ensure that at least the desired amount of amplification products is produced. In an exemplary embodiment, the desired amount may be selected based on an amount that will be sufficient to perform subsequent sequencing reactions. In at least one embodiment in accordance with the present teachings, therefore, quantification of the primer extension product is not performed after the amplification reaction and before the sequencing reaction.

Magnetic capture of the amplified sample can also allow conventional cleaning steps to be avoided. For example, by capturing only the amplified strands, leftover or unreacted primers and/or nucleotides can be left behind in the container in which the amplification reaction took place. Therefore, in at least one embodiment of the present teachings, additional steps to remove leftover or unreacted primers and/or nucleotides, for example, by using reagents such as exoSAP, for example, are not used. This can reduce the amount of time of the overall sample preparation process, reduce costs, and reduce the number and amount of reagents used.

Strands 121 and 131 can be denatured and subjected to a sequencing reaction to generate families of sequencing reaction products or sequencing ladders. In FIG. 1, step C a forward sequencing primer 160 can hybridize to strand 121 and be extended to form sequence strand 161 (shown at step D). A reverse sequencing primer 170 can hybridize to strand 131 and be extended to form sequence strand 171 (shown at step E). In some embodiments of the invention, only a forward sequencing primer 160 is present in the sequencing reaction. In other embodiments only a reverse sequencing primer 171 is present and in yet other embodiments, both forward sequencing primer 160 and reverse sequencing primer 170 are present in the sequencing reaction. In at least one embodiment, when the sequencing reaction uses dideoxynucleotides, multiple sequence strands 161 and 171 having various lengths are formed. Thus, the families of sequencing reaction products comprising strands 161 and 171 comprise a plurality of various length sequence strands of the sequencing ladders. Capture substrates 125, along with attached strands 121, may be separated from the families of sequencing reaction products using a magnet 150 so that only the sequencing ladders comprising the created strands 161 and 171 are left behind in step C. While not wishing to be bound by theory, it is believed that most of strands 131 will rehybridize with strands 121 after formation of the sequencing ladders. It may be possible that a small number of strands 161 and 171 are also removed with the removal of the capture substrates 125 and attached hybridized strands 121 and 131. However, due to the sequencing reaction, strands 161 and 171 can greatly outnumber strands 121 and 131, so that a small percentage of strands 161 and 171 may be removed without negatively impacting the ability to perform sequence detection. For example, for a sequencing reaction that proceeds through 40 cycles, each of strands 121 and 131 may generate up to 40 strands 161 and 171, respectively. Therefore, the much greater number of strands 161 and 171 can ensure that a sufficient amount of strands 161 and 171 will not be captured and will remain after removal of the capture substrates 125 therefrom.

In at least one embodiment, forward sequencing primer 160 is attached to a prey moiety 162. According to at least one embodiment of the present teachings, prey moiety 162 comprises a polynucleotide sequence. Similarly, in at least one embodiment, reverse sequencing primer 170 is attached to a prey moiety 172, which also can comprise a polynucleotide sequence. In at least one embodiment, prey moiety 162 and prey moiety 172 can comprise distinct polynucleotide sequences.

When prey moiety 162 and prey moiety 172 comprise distinct polynucleotide sequences, they can be separated using differing capture compounds. For example, in step D of FIG. 1, capture substrate 165 comprising capture compound 163, which is complementary to prey moiety 162, can be used to selectively remove the formed sequence strands 161 from the sequencing reaction product mix. Likewise, in step E of FIG. 1, capture substrate 175, which comprises capture compound 173, can selectively remove the formed sequence strands 171 from the sequencing reaction product mix. This selective removal using complementary polynucleotide capture compounds may be referred to as hybridization based removal or pull-out. Hybridization based pull-out can enable the selective elution of a desired product for subsequent reactions and/or analysis, such as, for example, capillary electrophoresis. As one skilled in the art would recognize, the capture compound may bind to the prey moiety via DNA-DNA hybridization, DNA-RNA hybridization, RNA-RNA hybridization, or any other combination comprising natural and synthetic bases. The capture compound and/or the prey moiety may have a nucleotide length of 15-25 bp.

In accordance with at least one embodiment of the present teachings, capture substrate 165 and capture substrate 175 may be magnetic so as to be attracted to magnet 150. In various exemplary embodiments, the substrates 165 and 175 may comprise magnetic particles, beads, or the like. For example, the substrates 165 and 175 may be coated magnetic particles. The coating of the magnetic particle of substrates 165 and 175 may be a plastic coating, such as, but not limited to, polystyrene. The particle may comprise a linker compound on the surface, such as, but not limited to a polyethylene glycol (PEG) linker. The PEG linker may have a length in the range of about 3 to about 20 PEG monomer units. The PEG linker on the particle may be covalently bound to the capture compound, which is the specific hybridization sequence having a nucleotide sequence of 15-25 bp length. The PEG linker on the particle may be covalently bound to the capture compound at the 3′ or 5′ end of the capture compound sequence. The specific hybridization sequence can hybridize to the complementary polynucleotide prey moiety attached to the sequencing primer. The particle may alternatively comprise an active group on the surface thereof, such as, for example, streptavidin. In an exemplary embodiment, the substrates 165 and 175 may comprise avidin, which forms a strong non-covalent bond with biotin. In at least one embodiment, the particle may have a dimension (e.g., diameter) ranging from about 1 μm to about 5 μm. According to at least one embodiment, the particle may comprise Dynal® magnetic beads (available from Life Technologies Corp.) which are 2.8 μm coated beads comprising a magnetic core.

Although the exemplary workflow described above with reference to FIG. 1 has the forward primer 120 as the capturable primer that binds with the capture compound 126 on the capture substrate 125, those having ordinary skill in the art would appreciate that the reverse primer 130 instead could be the capturable primer and enable specific binding to the capture compound 126 on the capture substrate 125. Ordinarily skilled artisans would understand how to modify the workflow described with reference to FIG. 1 for such a situation.

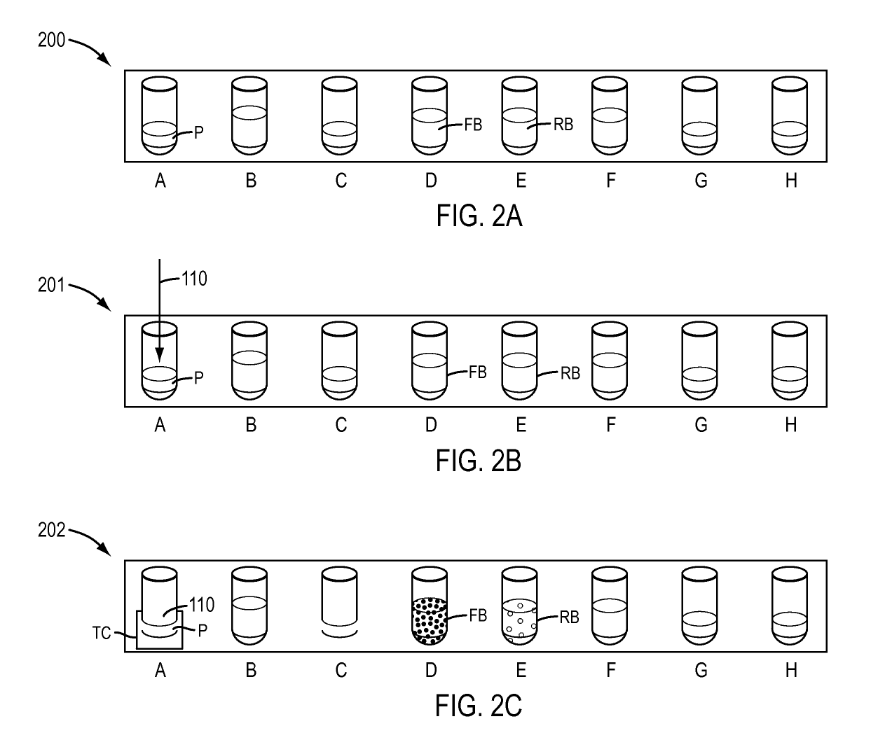

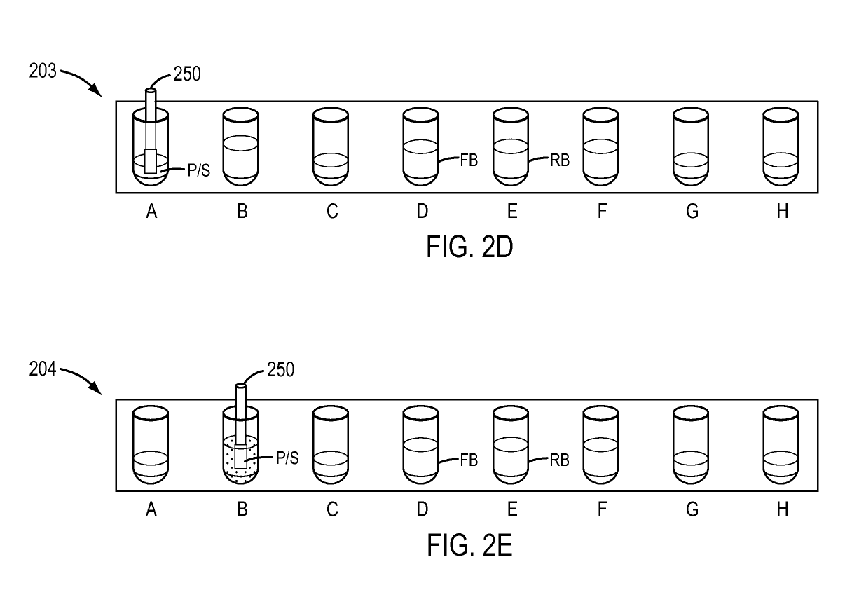

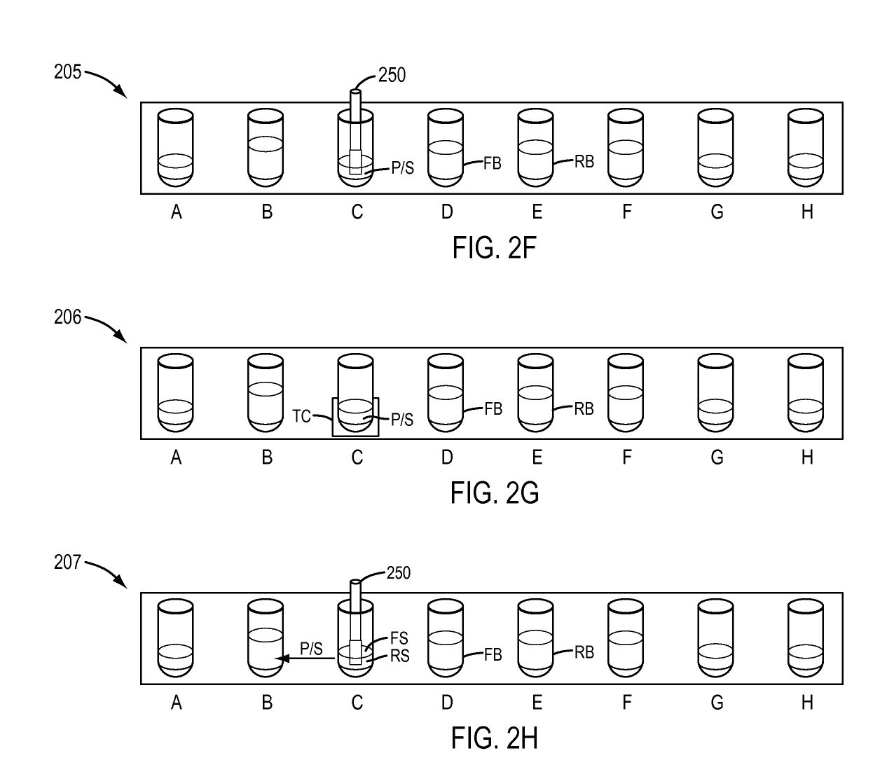

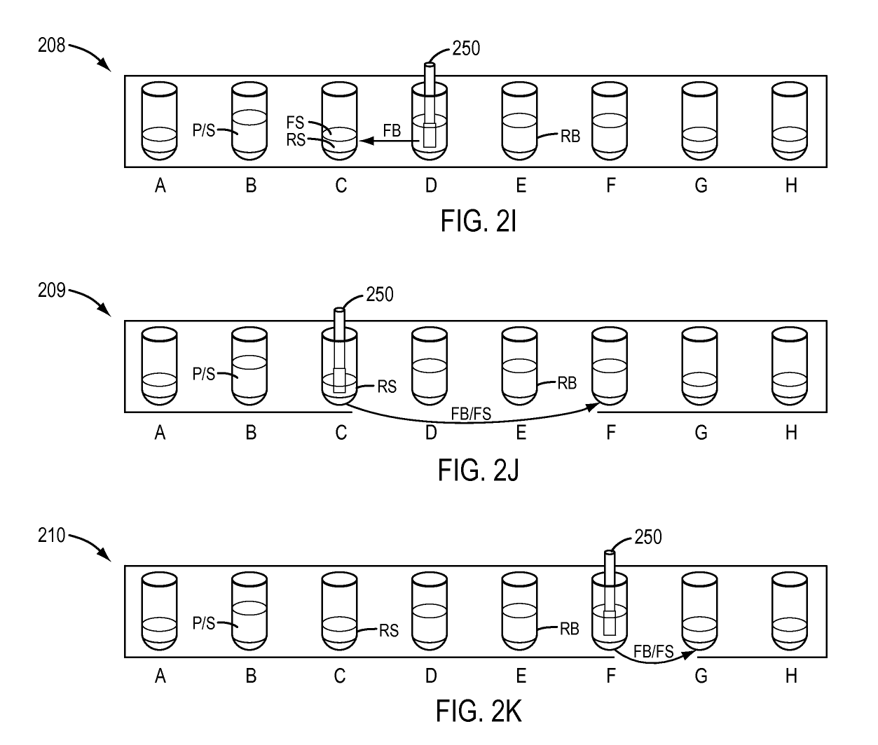

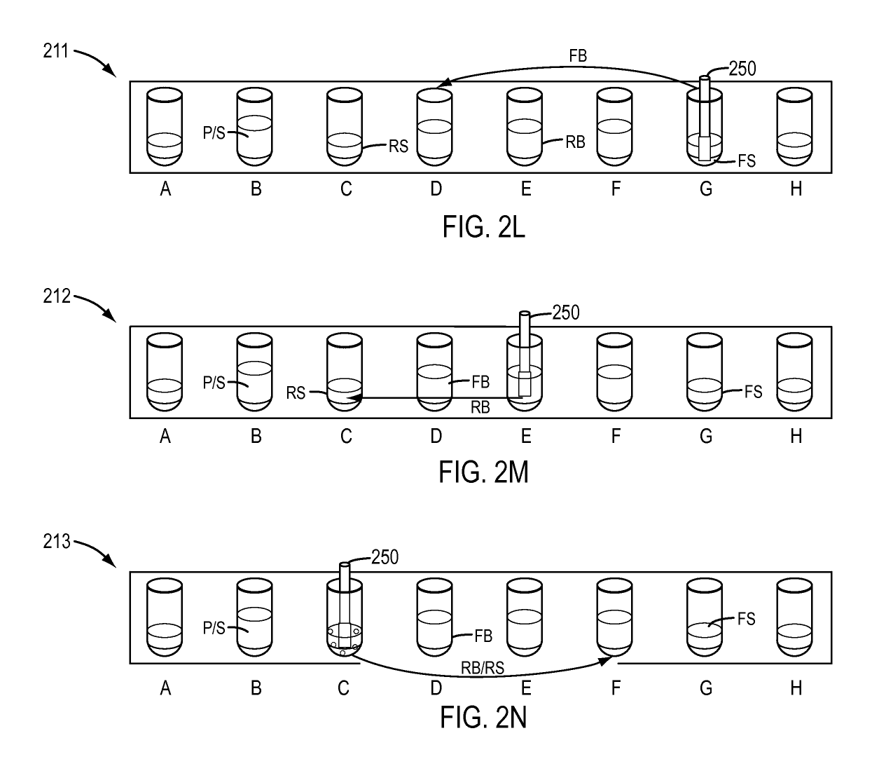

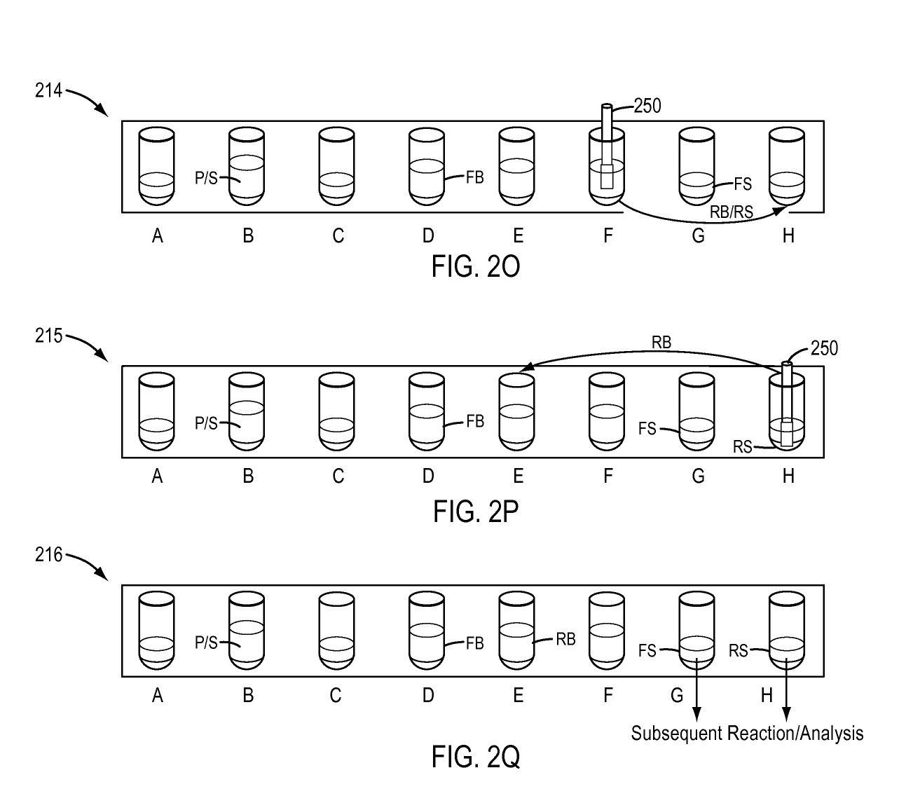

One exemplary embodiment for carrying out a workflow of the generalized nucleic acid sample preparation process described with reference to FIG. 1 is schematically shown in FIGS. 2A-2Q.

In step 200 shown in FIG. 2A, 8 containers labeled A-H are provided. The containers A-H may comprise, for example, microcentrifuge tubes, sample tubes, test tubes, wells in a well plate, or any other type of container for use in nucleic acid sample preparation known to those ordinarily skilled in the art. As depicted in FIG. 2A, the containers A-H comprise tubes. Tube A contains dried-down (e.g., lyophilized) reagents for PCR, magnetic particles P with a forward primer, like forward primer 120 of FIG. 1, attached, and reverse primers, like reverse primers 130 of FIG. 1. In other embodiments, the reagents may comprise hydrated non-lyophilized reagents. These reagents may be added to the container by the user, or provided in the container as a kit. The particles may comprise, for example, a bead comprising a magnetic core covered by a plastic coating, such as polystyrene. The particles may also comprise an active group on the surface thereof, such as, for example, streptavidin. In at least one embodiment, the particles may have a dimension (e.g., diameter) ranging from about 1 μm to about 5 μm. According to at least one embodiment, the capture substrate may comprise Dynal® magnetic beads (available from Life Technologies Corp.) which are 2.8 μm coated beads comprising a magnetic core. As one skilled in the art would recognize, the capture substrate may have a variety of configurations and be made from a variety of materials, and the specific examples above are non-limiting and exemplary only.

In an alternative exemplary embodiment, the magnetic particles P could have the reverse primers attached thereto and the forward primers could be provided separate from the magnetic particles P in the tube A. Those of ordinary skill in the art would understand how to modify the remaining steps for such a situation. Tube B contains a wash solution, such as, for example, a solution of 20 mM Tris-HCl (tris(hydroxymethyl)aminomethane hydrochloric acid) and 0.1% Tween (polyoxyethylene (20) sorbitan monolaurate). Tube C contains reagents for enabling a sequencing reaction, including forward and reverse sequencing primers like primers 170, 160 described in FIG. 1. Tube D contains magnetic particles FB for hybridization based pull-out of a forward sequencing reaction. Tube E contains magnetic particles RB for hybridization based pull-out of a reverse sequencing reaction. In at least one embodiment, magnetic particles FB and RB may comprise particles like those used for the capture substrates described above. By way of non-limiting example, the particles may be Dynal® magnetic beads. Tube F contains a wash solution, such as, for example, 20 mM Tris-HCl and 0.1% Tween. Tubes G and H, contain a denaturing compound, such as, for example, formamide (e.g., highly deionized formamide, Hi-Di™ Formamide available from Life Technologies Corporation). Tubes G and H can be used, for example, to hold the forward and reverse sequencing reaction products, respectively, for subsequent capillary electrophoresis.

In step 201 shown in FIG. 2B, a nucleic acid sample (depicted by the arrow) containing target nucleic acid 110 for which sequencing is desired is placed in tube A and allowed to mix with the contents that are predisposed in tube A. The combined contents of tube A may be mixed, for example, by the introduction of the sample 110 into tube A. In various exemplary embodiments, it may be desirable to mix the sample with the other contents in tube A; such mixing may occur by pipetting the sample liquid up and down, by vibrating the tube, and/or by agitating the contents in the tube A axially and/or rotationally or in any manner known to those skilled in the art. Although tube A is described above as containing the predisposed PCR reagents, magnetic particles, and primers, it is within the scope of the present teachings that all of the contents to support an amplification reaction in accordance with the present teachings could be introduced at the time of sample introduction during step 201.

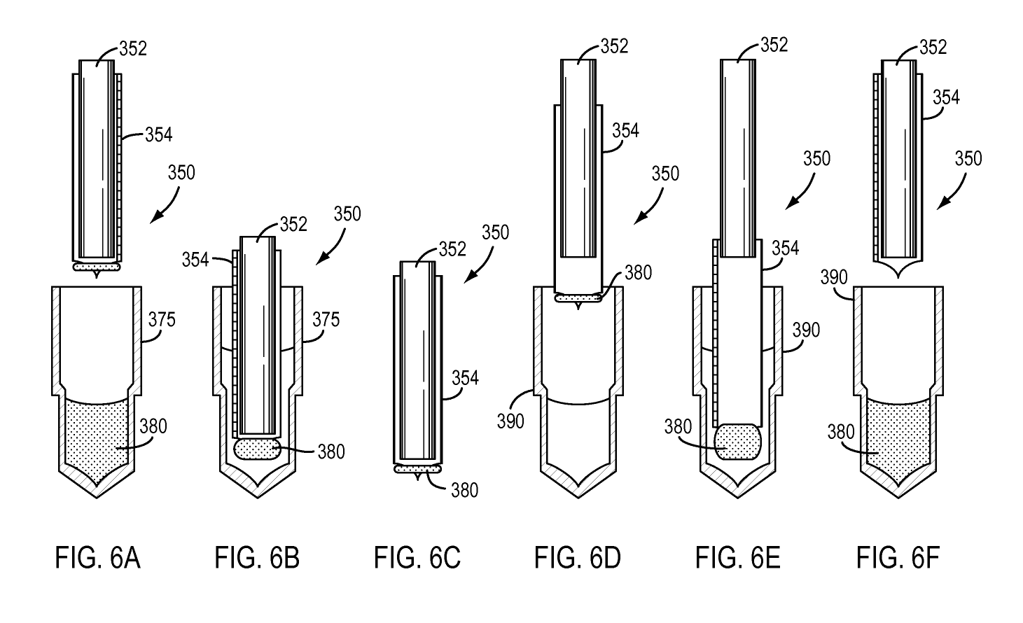

In the following step 202, shown in FIG. 2C, the target nucleic acid in sample 110 is subjected to an amplification reaction, for example, using thermal cycling as schematically represented by TC in the figure. For example, the amplification reaction may proceed substantially as described with reference to step A of FIG. 1. The amplification reaction of step 202 produces amplified sample, which comprises hybridized forward and reverse amplification strands, like the hybridized strands 121 and 131 shown in step B of FIG. 1, attached to the magnetic particles P. The particles with the hybridized strands are depicted as P/S in FIG. 2D. A magnet 250, such as, for example, a magnetic rod contained substantially concentrically within and independently moveable in an axial direction relative to non-magnetic (e.g., plastic) sheath, may be inserted into tube A, as shown in step 203 of FIG. 2D, to attract the magnetic particles P/S with the hybridized forward and reverse amplification strands attached thereto. Those having ordinary skill in the art are familiar with such plastic-sheathed magnetic rods used for magnetic particle-based assays and employed via various systems such as KingFisher 24 or KingFisher 96 (available from Thermo Electron Corp.) or a MagMAX™ Express or MagMAX™ Express-96 Particle Processor (available from Life Technologies Corp.). A further description of an exemplary embodiment of a sheathed magnetic rod and use of the same for particle transfer between two containers in accordance with the workflows described herein is set forth below with reference to FIG. 6.

By virtue of the magnet 250 attracting the particles P/S, the strands attached to the particles and the strands hybridized to the strands attached to the particles (e.g., like hybridized strands 121 and 131) that formed during the amplification reaction at step 202 are able to be removed from the tube A, leaving the remainder of the amplification reaction constituents behind. The captured strands can be cleaned by moving the magnet 250 with the magnetic particles P/S attached thereto to tube B, as shown in step 204 in FIG. 2E. In tube B, unreacted nucleotides, polymerase, and primers may be separated and removed from the strands by washing the strands attached to magnetic particles P/S in the wash solution in tube B.

In step 205 shown in FIG. 2F, after separation of the unreacted nucleotides, polymerase, and primers from the amplified sample strands, the particles P/S can be moved from tube B to tube C via magnet 250. As mentioned above, tube C can contain (either by way of predisposition therein or introduction with the particles P/S), reagents including forward and reverse sequencing primers, like 160 and 170 of FIG. 1, for generating sequencing ladders of the amplified sample strands introduced into the tube C by deposition of the particles P/S thereto. At step 206, a sequencing reaction may then take place in tube C, which, for example, may rely on a thermal cycling reaction as schematically depicted as TC in FIG. 2G. For example, a sequencing reaction using dideoxynucleotides to generate forward sequencing ladder FS and reverse sequencing ladder RS, each of which comprises multiple sequence strands of various lengths, like strands 161 and 171 described above with reference to step C of FIG. 1, may be performed at step 206.

Following the sequencing reaction of step 206, a magnet 250 may again be introduced into tube C to attract the particles P/S and return them to tube B, leaving the forward sequencing ladder FS and the reverse sequencing ladder RS in tube C, as shown in step 207 in FIG. 2H.

In step 208 of FIG. 2I, the magnet 250 may be inserted into tube D to pick up the forward sequencing reaction capture substrates FB and move them to tube C. Once moved to tube C, the capture substrates FB comprising capture compounds can hybridize with the prey moieties present on the strands of the forward sequencing ladder FS (the capture substrates FB with the hybridized forward sequencing ladder FS attached thereto is depicted as FB/FS in FIGS. 2J and 2K).

In step 209 of FIG. 2J, a magnet 250 attracts the capture substrates FB with the forward sequence ladder FS attached thereto (FB/FS) and moves them to the wash solution in tube F, as shown in step 210 of FIG. 2K. In tube F, unreacted fluorescently labeled dideoxynucleotides or other sequencing reagents can be washed out and the washed forward sequencing ladder FS transferred to tube G by using the magnet 250 to move the washed particles FB/FS from tube F to tube G. The washed forward sequencing ladder FS can then be selectively eluted in tube G, for example, by denaturing the hybridized prey moiety and capture compound to separate the forward sequencing ladder FS from the capture substrates FB, as shown at step 211 of FIG. 2L. The forward sequencing capture substrates FB can be transferred back to tube D from tube G via magnet 250, as shown in FIG. 2L, step 211.

Magnet 250 can next be used to transfer the reverse sequencing capture substrates RB from tube E to tube C, as shown at step 212 of FIG. 2M. At step 213 of FIG. 2N, the reverse sequence ladder RS in tube C can hybridize to the capture compound of reverse sequencing capture substrates RB. In step 213, the reverse sequencing capture substrates RB with reverse sequencing ladder RS attached thereto (RB/RS) are transferred to tube F using magnet 250. In tube F, the reverse sequencing ladder RS is washed to remove sequencing reagents, including fluorescently labeled dideoxynucleotides and polymerase. At step 214, the washed reverse sequencing ladder RS is transferred to tube H by using magnet 250 to move the washed particles RB/RS from tube F to tube H, as shown in FIG. 2O. The reverse sequencing ladder RS can be eluted into tube H by denaturing the hybridized prey moiety and capture compound to separate the reverse sequencing ladder RS from the reverse capture substrates RB. The reverse capture substrates RB can then be returned to tube E using magnet 250, as shown in step 215 in FIG. 2P, leaving the reverse sequencing ladder RS behind in tube H.

In step 216, isolated forward sequencing ladder FS and reverse sequencing ladder RS, respectively contained in tubes G and H, can then be subjected to further reactions and/or analysis. By way of example, they may be subjected to a detection technique, such as, for example, capillary electrophoresis, to determine the base calls of the sequences as those ordinarily skilled in the art are familiar with.

As one skilled in the art would readily appreciate, hybridization based pull-out as described herein allows for multiplexing of the samples. For example, multiple target nucleic acid samples may be amplified and placed in a single container. Using differing prey moieties for each sequencing primer, differing capture compounds can be used to selectively separate each distinct sequencing reaction product. Although the examples above describe a system comprising two families of sequencing reaction products, i.e., the forward and reverse sequencing ladders FS and RS, one ordinarily skilled in the art would recognize that the system may be modified to capture any number of families of sequencing reaction products, ranging from one to more than one, as desired. In various exemplary embodiments, for example, up to 12 or more families, for example, from 4 to 10 families, of sequencing reaction products may be simultaneously processed according to the present teachings.

In accordance with at least one embodiment, the various transfer steps from one container to another may be performed manually. In at least one further embodiment, one or more of the transfer steps may be automated. For example, an automated, robotic magnetic particles processor, such as, for example, a KingFisher 24 or KingFisher 96 (available from Thermo Electron Corp.) or a MagMAX™ Express or MagMAX™ Express-96 Particle Processor (available from Life Technologies Corp.) may be used to automate one or more steps in the sample preparation method depicted and described with reference to the exemplary embodiment of FIGS. 2A-2Q.

Magnetic particles processors may comprise a plurality of magnetic rods that fit within a non-magnetic (e.g., plastic) sheath or tip separating the magnet from the sample. The magnetic rods and non-magnetic sheaths or tips may move axially independently relative to each other to enable the magnetic force to be distanced from the sample, e.g., by virtue of retraction of the magnetic rod in the sheath, or to act on the sample, e.g., by virtue of advancing the end of the magnetic rod closest to the sample closed to an end of the sheath closest to the sample. The magnetic rods may be controlled via a program according to the desired reaction parameters.

An exemplary sheathed magnetic rod and use of the same for transferring magnetic particles is shown in the schematic depiction of FIGS. 6A-6F. As shown, the sheathed magnet 350 includes a magnetic rod 352 concentrically disposed within a non-magnetic (e.g., plastic) sheath 354. The sheathed magnet 350 may be positioned over a source container 375 containing magnetic particles 380 for which transfer to another destination container 390 is desired. In FIG. 6B, the sheathed magnet 350 may be inserted into the source container 375, with the magnetic rod 352 in an axial position relative to the sheath 354 so that the magnetic force from the magnetic rod 352 can act on the magnetic particles 380 with sufficient strength to attract the particles toward the magnetic rod 352 and end of sheath 354 closest to the sample in the source container 375. With the magnetic particles 380 held via magnetic force to the end of the sheathed magnet 350, the sheathed magnet 350 may be removed from the source container 375, as shown in FIG. 6C, and then inserted into the destination container 390, as shown in FIG. 6D. When inserting the sheathed magnet 350 into the destination container 390, the relative axial position of the sheath 354 and the magnetic rod 352 may be altered so that the magnetic rod 352 is retracted into the sheath 354 and the end of the sheath 354 to which the particles 380 are held is positioned within the container 390. At a sufficient distance of retraction, the magnetic force of the magnetic rod 352 will no longer act on the particles 380 with sufficient strength for the particles 380 to be held relative to the end of the sheath 354 and the particles 380 will be released from the sheathed magnet 350 into the container 390, as shown in FIG. 6E. Thereafter, the sheathed magnet 350 can be removed from the container 390 leaving the particles 380 behind in the container 390. As shown in FIG. 6E, the relative axial position of the sheath 354 and magnetic rod 352 may be repositioned after withdrawal so that the sheathed magnet 350 can again be used for magnetic particle transfer.

In at least one embodiment, a magnetic particles processor may be used during the amplification, sequencing reactions, and/or sequencing reaction purification.

By performing certain steps of certain embodiments of the present automatically, i.e., without requiring the operation, control, or other intervention of a human operator, nucleic acid sample preparation can be completed more rapidly. For example, by automating the sample preparation process with magnetic separation, the time for processing nucleic acid samples from start to finish may be significantly reduced, cutting the time to process the samples by up to half that of a conventional workflow that involves liquid transfer steps performed via pipetting for example. More specifically, and by way of non-limiting example, preparing 96 samples for sequencing can take about 100 minutes using a conventional manual liquid transfer workflow, whereas the workflow described herein using an automated magnetic particles processor can take about 55 minutes. In addition, automating the process with magnetic separation may also provide cost benefits by reducing the number of reagents required and/or the amount of reagents used.

In alternative exemplary embodiments, more than one sequencing ladder may be removed by using a capture compound that selectively binds with the prey moiety of each sequencing ladder. In at least one embodiment, the prey moiety may comprise multiple prey moieties that can bind with multiple capture compounds.

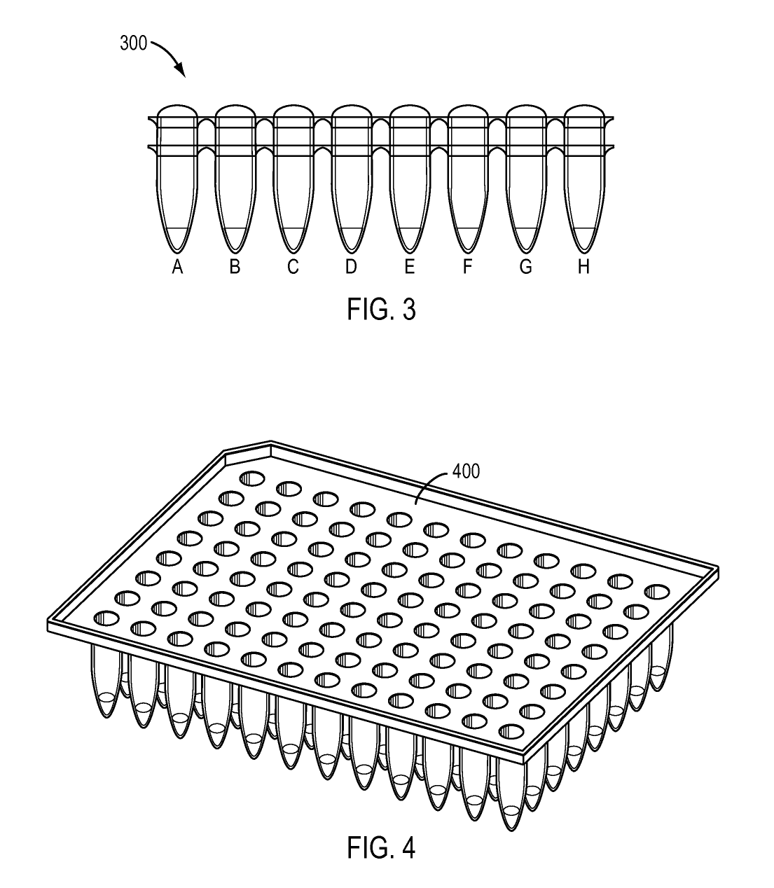

The present teachings further relate to a kit for nucleic acid sample preparation. In at least one embodiment, the kit may comprise multiple containers comprising reagents and solutions required for the sample preparation method described above with reference to the FIGS. 2A-2Q. For example, a strip of 8 tubes may comprise lyophilized reagents including magnetic particles comprising capture compounds, PCR primers, sequencing primers, and wash solutions. An example of a strip of tubes comprising lyophilized reagents is shown in FIG. 3.

In FIG. 3, strip 300 comprises tubes A-H. Tube A comprises forward and reverse PCR primers and magnetic particles respectively attached to at least one of the forward and reverse PCR primers. Tubes B and F each contains wash solutions, for example, wash solutions comprising Tris-HCl and Tween. Tube C comprises forward and reverse sequencing primers. Tubes D and E contains forward and reverse capture substrates, respectively, for hybridization based pull-out. Tubes G and H each contains a denaturing solution.

As one skilled in the art would recognize, the strip may comprise additional tubes if more samples are being prepared at the same time. For example, two additional tubes may be added for each additional sequencing ladder, with one tube containing capture substrates comprising a capture compound for the additional family of sequencing ladders, and one tube containing a denaturing solution for the cleaned additional family of sequencing ladders.

In at least one embodiment, a plate comprising multiple sets of containers, for example, multiple strips as above, may be provided. For example, in FIG. 4, a 96-well plate 400 is shown comprising the reagents described above, with each set of 8 containers containing potentially unique reagents if desired to permit preparation of differing samples. For the exemplary process described above, each 96-well plate could be used for 12 different nucleic acid samples.

In at least one alternative embodiment, a plurality of individual well plates 400 may be used, wherein each of the plurality of well plates contains one of the reagents described above, to simultaneously perform a plurality of different reactions. For example, eight 96-well plates may be used wherein each of the plates contains one of the contents described with respect to the eight tubes A-H described above with reference to FIGS. 2A-2Q. A magnetic rod assembly that includes a plurality of magnetic rods to act simultaneously on multiple wells of the well plates may also be used to carry out the workflow described above with respect to the eight tubes A-H described above with reference to FIGS. 2A-2Q. Those having ordinary skill in the art would understand that a 96-well plate is exemplary only and well plates having more or less wells may be utilized in accordance with the present teachings.

According to at least one embodiment of the present teachings, the reagents may be present in lyophilized form, or they may be present in solvents. The reagents may also be provided within the containers as part of a kit. In other embodiments, the reagents may be added to the containers just prior to use. The reagents may be added either manually or automatically, such as, for example, with an automated or robotic loader.

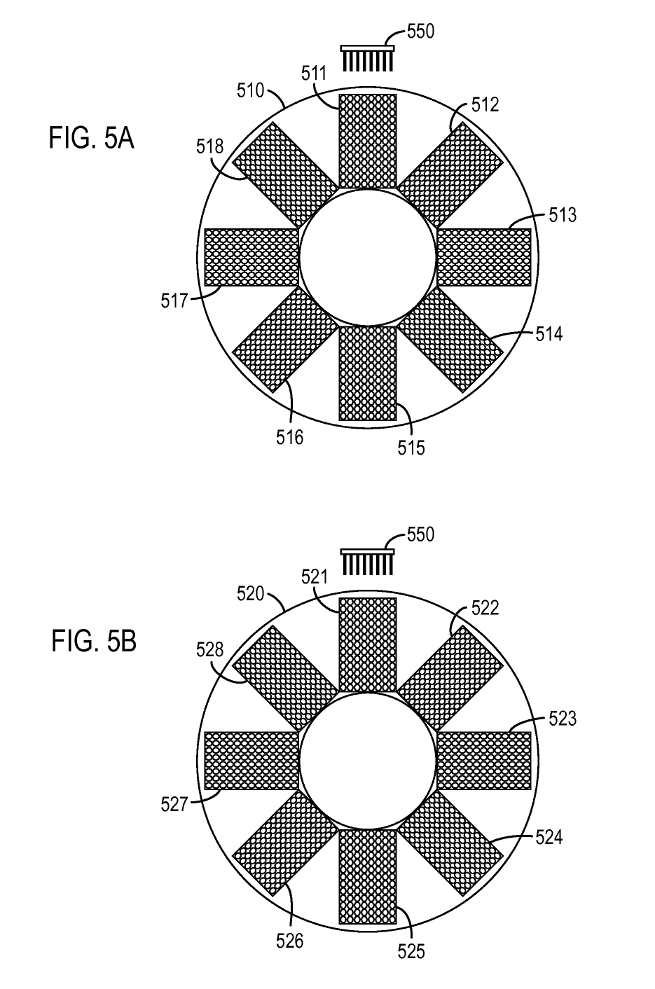

In at least one embodiment in accordance with the present teachings, a magnetic particles processor can be used to automate a sample preparation workflow similar to that described above with reference to FIGS. 2A-2Q with one difference being that instead of individual tubes at each station and one magnet, a plurality of well plates are used for each step of the process and a multiple magnet assembly is used for transferring between each well plate station. FIGS. 5A and 5B depict a schematic representation of the carousels of two MagMAX™ Express-96 Particles Processors used in tandem in accordance with one exemplary embodiment. A first particles processor has a carousel 510 containing eight 96-well plates 511-518. In at least one embodiment, the first carousel 510 can be set up for sample amplification and purification. Plate 511 is a tip plate, which may contain a wash solution to clean the tips covering magnetic rod assembly 550. Plate 512 may contain magnetic capture substrates used to capture and move the amplified target sample (e.g., like capture substrates 125). Magnetic rod assembly 550 may move the substrates from plate 512 to plate 513, which contains reagents for amplifying the target sample. Amplification may then occur in plate 513, after which the magnetic rod assembly 550 may transport the magnetic capture substrates carrying the amplified target sample from plate 513 to one or more plates containing a wash solution. For example, plate 514 may contain a wash solution that removes unreacted primers and/or nucleotides. The washed sample held by the magnetic capture substrates may then be transported by the magnetic rod assembly 550 to plate 515 which contains sequencing reaction reagents. Plates 516-518 may contain additional wash solutions or they may be empty or not present when the process does not require their use.

A second magnetic particles processor, shown in FIG. 5B, may be adapted for the sequencing reaction and/or purification of the sequencing reaction products. Carousel 520 may carry up to eight 96-well plates 521-528, each of which contain a reagent or solution for performing the sequencing reaction and/or purification of the sequencing reaction products. Plate 521 may be a tip plate where the tips covering the magnetic rods of magnetic rod assembly 550 may be rinsed or washed between actions. Plate 522 can be a sequencing plate where sequencing reactions are carried out, as described above with reference to step C of FIG. 1 using forward and reverse sequencing primers. Alternatively, the sequencing reactions may take place in plate 512 of the first carousel 510 and then transferred to the second carousel 520 and placed where plate 522 is located for subsequent purification of the sequencing reaction products, as will be described. After the sequencing reaction has taken place, plate 523 can be used to recover the magnetic capture substrates with the amplified strands attached (e.g., like capture substrates 125 with the strand 121 and/or hybridized strands 121 and 131 attached) by moving the magnetic capture substrates in plate 522 to plate 523. Plates 524 and 525 may respectively contain forward and reverse sequencing reaction capture substrates (e.g., like substrates 165 and 175 of FIG. 1) for capturing and separating the sequencing reaction products using hybridization based pull-out in plate 522. The capture substrates may be transferred from plates 524 and 525, respectively, to plate 522 using the magnetic rod assembly 550. Plate 526 may contain a wash solution for washing the forward and reverse reaction products before they are transferred by the magnetic rod assembly 550 to their respective elution plates 527 and 528. Thus, after hybridization based pull-out in plate 522, the magnetic rod assembly 550 may be used to move the respective forward and reverse capture substrates with the forward and reverse sequencing ladders respectively attached thereto from the plate 522 to the wash plate 526 and then to the respective elution plates 527 and 528. As one skilled in the art would readily appreciate, the arrangement and purpose of each plate may be modified within the spirit of the present disclosure to account for different reaction parameters.

The automated magnetic particles processor may operate by rotating the carousel to position each plate within the reach of the magnetic rod assembly. Alternatively, the magnetic rod assembly may be moved to each of the plates. Other robotic systems may also be used for the movement and manipulation of the samples and/or containers.

Although various embodiments are described with reference to MicroSEQ™ and dideoxy sequencing techniques, it should be understood that the nucleic acid sample preparation methods and workflow principles can be applied to other techniques. The preparation methods and workflow principles according to the present teachings can be adapted for other applications requiring separation, purification, and/or manipulation of nucleic acid samples. Those ordinarily skilled in the art would understand how to make modifications to the lengths, design, sequences, etc., of the capturable primers, specific binding pairs, primer moieties, prey moieties, capture compounds, and/or capture substrates to optimize applicability in other sequencing systems/techniques, as well as other applications requiring the separation, purification, and/or manipulation of nucleic acid samples.

According to exemplary embodiments of the present invention, one or more parameters pertaining to the PCR reactions as discussed in the foregoing may advantageously be optimized. For example, regarding setting up the PCR reaction, the concentration of primers (including whether to biotinylate one or both primers), the percentage of biotinylated primers versus non-biotinylated primers, the number of cycles performed, and whether to intentionally make on reagent limiting (e.g., by limiting an amount of forward primer so it runs out first, one may be able to control an amount of PCR product entering the subsequent purification reactions) may be optimized. One may also examine and optimize the effect of input template concentration on downstream purification/sequencing. For another example, regarding purifying the PCR product, the amount of beads (e.g., magnetic beads) to include, the binding times and conditions, the wash times and conditions (including the buffer composition and mixing speed), and whether to elute or add magnetic beads with the PCR product that may still be bound directly to sequencing reaction may also be optimized.

According to exemplary embodiments of the present invention, one or more parameters pertaining to the sequencing reactions as discussed in the foregoing may advantageously be optimized. For example, regarding setting up the sequencing reaction, the amount of primer to use (including for single or dual sequencing reactions), how much master mix to use, whether to include beads in the sequencing reaction, and the number of cycles of sequencing reaction to perform may be optimized. For another example, regarding the purifying of the sequencing reaction, how much beads to include, how to couple the beads (including whether or not saturated and whether direct or indirect), the conditions and time for hybridization and washing, the conditions for elution, and whether to pursue sequential sequence purification may also be optimized.

For example, exemplary parameters according to embodiments of the invention may include one or more of the following: DNA extraction may be based on Dynal® MyOne Silane beads; PCR conditions may include using GeneAmp® Fast Master Mix (Life Technologies, Inc.), 10 ng per reaction of input template DNA, about 33% of forward primers being biotinylated with a total primer concentration of about 50 nM, and performing 40 cycles rather than 30 cycles under non-fast thermal cycling conditions; PCR purification may include using MagMAX™ Express-96 (Life Technologies, Inc.), 5 microliters of streptavidin-coated beads, 2× washes with low TE plus 0.1% Tween20, and a concluding script with magnetic beads having PCR product attached deposited in a well with sequencing mixture; sequencing may include non-fast cycling conditions and may be based on sequencing primers containing a hybridization based pullout (HBP) tag; and sequencing purification may include using MagMAX™ Express-96, 5 microliters of magnetic beads coupled to capture oligonucleotides against HBP sequence, hybridization at about 55° C. for about 8 minutes without 94° C. melting, 2× washes with low TE plus 0.1% Tween20, and elution in 50% formamide.

It should also be understood that the various methods and steps described in the foregoing description may all be implemented with appropriate software and hardware components. In particular, the methods and steps described herein can be implemented by executing a computer program using a computer or microprocessor embedded in an automated fluid-handling apparatus (or otherwise in communication with such an apparatus) to control how and when the apparatus manipulates containers, places or removes fluids or particles in such containers, activates other features such as thermal cycling for a particular container, and moves or controls other components necessary to perform the steps. For example, in the case of an automated fluid-handling apparatus including a magnet such as described herein, such a computer program could be used to automatically control the movement of the magnet into and out of a given container and between any two distinct containers, so as to allow transfer of magnetic material from one container to the other. A suitable computer program for performing the various methods and steps described herein could be written in various languages, such an assembly language or a high-level language such as C, C++, Java, etc., and a person of ordinary skill in the art, given the benefit of the foregoing description describing the steps to be performed, could implement such a program.

The workflow described above may be used to automate sample preparation for a wide variety of applications, including but not limited to Human Leukocyte Antigen (HLC) analysis, MicroSeq® kit analysis, cancer gene sequencing or inherited gene sequencing.

### EXAMPLES

Using control E. coli DNA (from a MicroSeq® ID kit, Life Technologies, Inc.) as sample, 10 ng DNA is used in the workflow as described above and as illustrated in FIGS. 2A-Q.

### Example 1

In the amplification reaction, purification and normalization of a PCR reaction is performed as depicted in steps 201, 202, 203, and 204 of the workflow illustrated above. In the PCR reaction, GeneAmp® Fast Master Mix (Life Technologies, Inc.) is used; 33% of the forward primers is biotinylated; and forty cycles of PCR are performed. Purification is performed on a MagMAX™ Express-96 Particle Processor (Life Technologies, Inc.), using 0.2 mg of streptavidin-coated magnetic beads (Dynal® magnetic beads, Life Technologies, Inc.), and the collected magnetic beads containing bound PCR product are washed twice in TE buffer at pH 8.8.

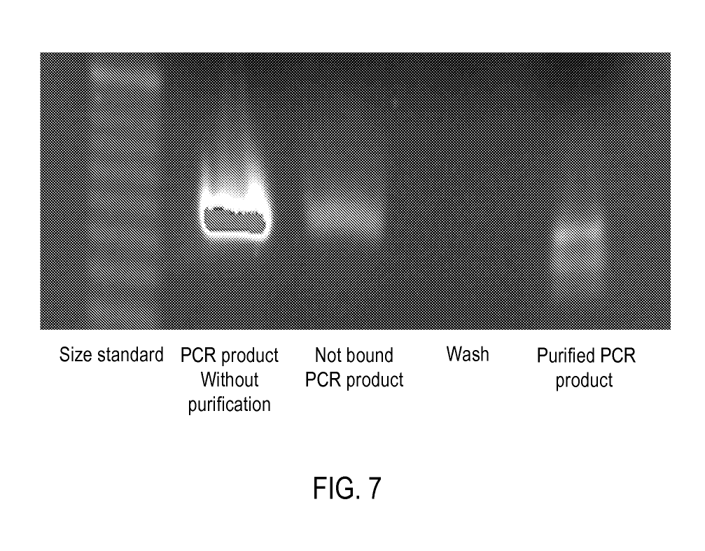

Aliquots of the crude PCR product, the amplification reaction mixture after removal of the magnetic beads containing bound PCR product, used wash solution, and the PCR product isolated after the wash step 204, are separated in a gel electrophoretic experiment. The results are shown in FIG. 7, and demonstrate that suitably clean PCR product of sufficient quantity for subsequent reaction and sequencing is afforded by the methods described above. The product is desalted and transferred to a desired location without manual handling. The gel electropherogram also demonstrates that there is little loss in the wash solution or in the capture step after PCR amplification and transferral away from the amplification reaction mixture.

### Example 2

The above bead-bound purified PCR product of control E. coli DNA, produced as described in Example 1, is treated according to workflow steps 206, 207, 208, 209, 210, and 211 wherein only a forward sequencing primer is used in the sequencing reaction 206. The other components of the sequencing reaction include BigDye Terminator V1.1 Cycle Sequencing Kit (Life Technologies, Inc.). Specific conditions include 2.5× Ready Reaction mix (16 μl); 4 μl primers (approximately 3.5-4 pmol), 20 μl water for a total volume of 40 μl. Cycle sequence: a. Rapid thermal ramp to 96° C.; b. 96° C. for 1 min; c. Repeat the following for 40 cycles: i. Rapid thermal ramp to 96° C.; ii. 96° C. for 10 seconds; iii. Rapid thermal ramp to 50° C.; iv. 50° C. for 5 seconds; v. Rapid thermal ramp to 60° C.; and vi. 60° C. for 4 minutes.

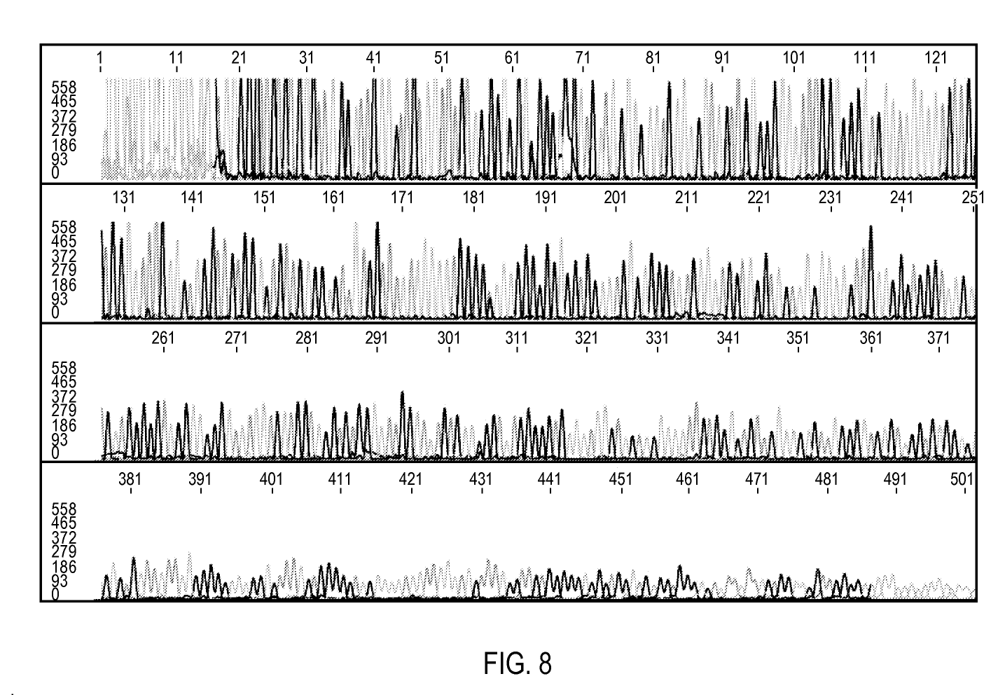

The forward sequence ladder FS thus obtained is captured by capture substrate FB which is a magnetic particle (Dynal® magnetic beads, Life Technologies, Inc.), functionalized with a polyethylene glycol linker which in turn is covalently bound to the 3′ end of a specific hybridization tag of about 15 to about 25 bp in length which forms the capture compound moiety of FB. This hybridizes with a complementary prey moiety of the forward sequence ladder FS, to effect the capture of step 208. After transference of bound forward sequence ladder FS/FB, washing with buffer, and denaturation as in steps 209, 210, and 211, the isolated forward sequence ladder FS is sequenced, using a 3130 Genetic Analyzer Sequencer (Life Technologies, Inc.) with POPE separation polymer (Life Technologies, Inc.). The results are shown in FIG. 8, and demonstrate very good quality data in the electropherogram.

### Example 3

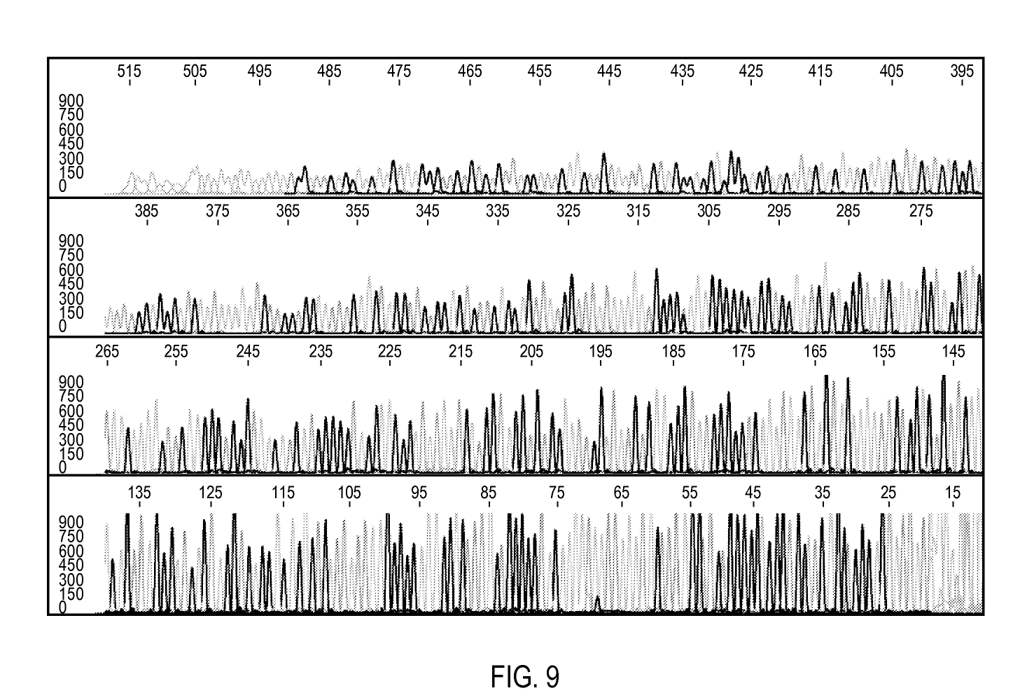

The above bead-bound purified PCR product of control E. coli DNA, produced as described in Example 1, is treated as described above according to workflow steps 206, 207, 212, 213, 214 and 216, wherein only a reverse sequencing primer is used in the sequencing reaction 206. The sequencing reaction is run under the same conditions as described in Example 2. The reverse sequence ladder RS thus obtained is captured by capture substrate RB which is a magnetic particle (Dynal® magnetic beads, Life Technologies, Inc.), functionalized with a polyethylene glycol linker which in turn is covalently bound to the 3′ end of a specific hybridization tag of about 15 to about 25 bp in length which forms the capture compound moiety of RB. This hybridizes with a complementary prey moiety of the forward sequence ladder RS, to effect the capture of step 213. After transference of bound forward sequence ladder RS/RB, washing with buffer, and denaturation as in steps 213, and 214, the isolated reverse sequence ladder RS is sequenced, using a 3130 Genetic Analyzer Sequencer (Life Technologies, Inc.) with POPE separation polymer (Life Technologies, Inc.). The results are shown in FIG. 9, and demonstrate very good quality data in the electropherogram.

### Example 4

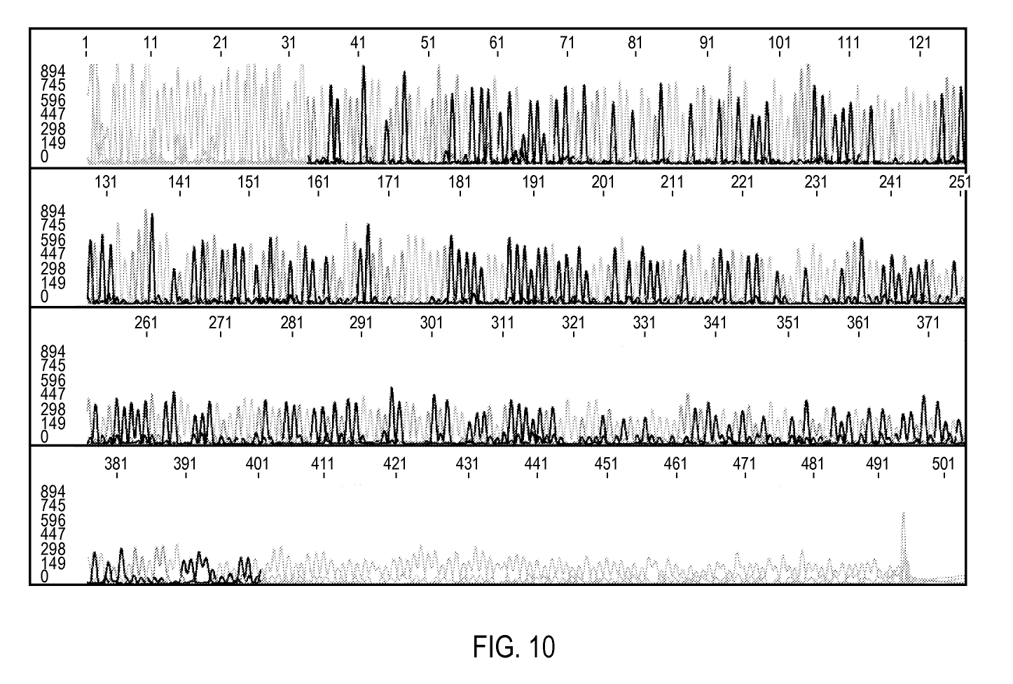

The above bead-bound purified PCR product of control E. coli DNA, produced as described in Example 1, is treated according to workflow steps 206, 207, 208, 209, 210, and 211 wherein both a forward sequencing primer and a reverse sequencing primer are used in the sequencing reaction 206. The sequencing reaction is run under the same conditions as described in Example 2. The forward sequence ladder FS thus obtained is captured by capture substrate FB, which is configured as in the FB of Example 2. After transference of bound forward sequence ladder FS/FB, washing with buffer, and denaturation as in steps 209, 210, and 211, the isolated forward sequence ladder FS is sequenced, using the instrument and polymer of Example 2. The results are shown in FIG. 10, and demonstrate good quality data in the electropherogram.

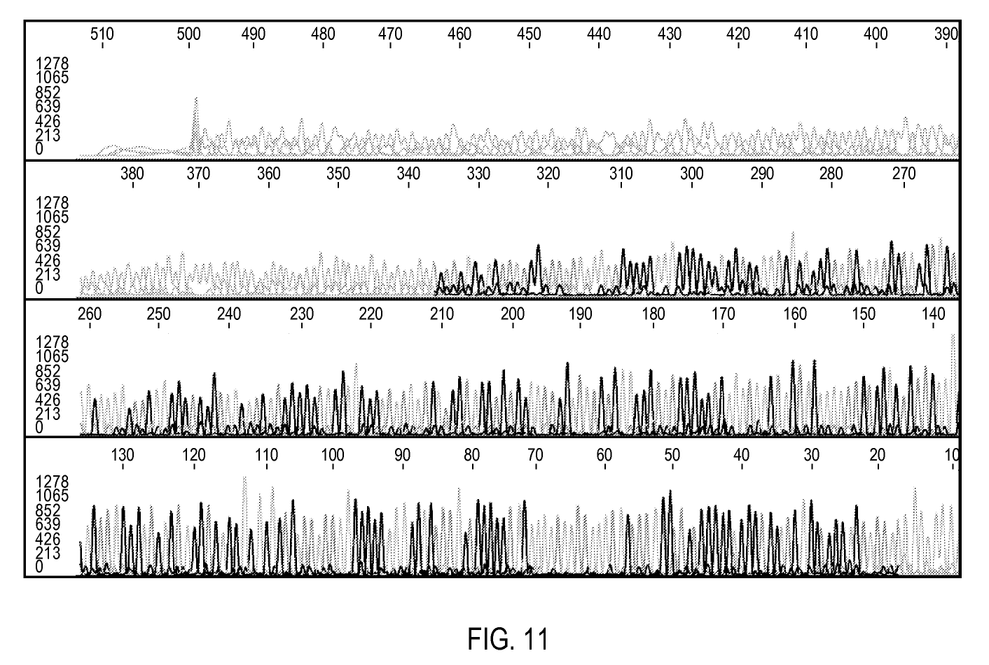

The further steps of workflow 213, 214, and 215 are carried out and permit isolation of reverse sequence ladder RS, as described above. Reverse sequence RS is sequenced using the instrument and polymer of Example 2. The results are shown in FIG. 11, and demonstrate good quality data in the electropherogram.

While the principles of the present teachings have been described in connection with specific embodiments of nucleic acid sample preparation and sequencing platforms, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

1-50. (canceled)

51. A method for automatically preparing nucleic acid samples for nucleic acid sequencing with high-throughput, comprising:
placing, in an apparatus configured to manipulate fluids and magnetic particles, (1) a reagent for PCR, a magnetic particle, a forward primer, and a reverse primer in a first container, (2) a wash solution in a second container, (3) forward and reverse sequencing primers in a third container, (4) a forward sequencing capture substrate in a fourth container, (5) a reverse sequencing capture substrate in a fifth container, (6) a wash solution in a sixth container, (7) a denaturing agent in a seventh container, and (8) a denaturing agent in an eighth container;
loading a nucleic acid sample including target nucleic acid in the first container; and
allowing the nucleic acid sample to mix with the reagent for PCR, magnetic particle, forward primer, and reverse primer in the first container.

52-54. (canceled)

55. The method of claim 51, wherein at least one of the forward primer and the reverse primer is attached to the magnetic particle.

56-60. (canceled)

61. The method of claim 55, further comprising automatically subjecting the target nucleic acid to an amplification reaction using thermal cycling to produce an amplified sample comprising hybridized forward and reverse amplification strands attached to the magnetic particle.

62. The method of claim 61, further comprising automatically inserting a magnet into the first container to attract the magnetic particle to which are attached the hybridized forward and reverse amplification strands.

63-65. (canceled)

66. The method of claim 62, further comprising:
automatically transferring the amplified sample comprising hybridized forward and reverse amplification strands attached to the magnetic particle from the first container to the second container with the magnet; and
washing the amplified sample comprising hybridized forward and reverse amplification strands attached to the magnetic particle using the wash solution in the second container to remove unreacted nucleotides, polymerase, and/or primers that may be on the hybridized forward and reverse amplification strands.

67-68. (canceled)

69. The method of claim 66, further comprising:
automatically transferring the forward sequencing capture substrate from the fourth container to the third container with the magnet; and
hybridizing the forward sequencing capture substrate with prey moieties present on the forward sequencing ladders of the amplified sample in the third container.

70-75. (canceled)

76. A method for increasing nucleic acid sample preparation throughput, comprising:
placing, in an apparatus configured to manipulate fluids, (1) a reagent for PCR, a magnetic particle, a forward primer, and a reverse primer in a first container, the forward primer being attached to the magnetic particle, and (2) a wash solution in a second container;
loading a nucleic acid sample including target nucleic acid in the first container;
mixing the nucleic acid sample with the reagent for PCR, magnetic particle, forward primer, and reverse primer in the first container by at least one of moving a pipetting device for pipetting the nucleic acid sample up and down the first container, vibrating the first container, and agitating the first container axially and/or rotationally;
automatically subjecting the target nucleic acid to an amplification reaction using thermal cycling to produce an amplified sample comprising hybridized forward and reverse amplification strands attached to the magnetic particle;
automatically inserting a magnet into the first container to attract the magnetic particle to which are attached the hybridized forward and reverse amplification strands; and
automatically transferring the amplified sample comprising hybridized forward and reverse amplification strands attached to the magnetic particle from the first container to the second container with the magnet.

77. The method of claim 76, wherein the magnet comprises a magnetic rod contained substantially concentrically within a non-magnetic sheath.

78. The method of claim 77, wherein the magnetic rod is independently moveable in an axial direction relative to the non-magnetic sheath.

79. A method for increasing nucleic acid sample preparation throughput, comprising:
placing, in an apparatus configured to manipulate fluids, (1) a reagent for PCR, a magnetic particle, a forward primer, and a reverse primer in a first container, (2) a wash solution in a second container, (3) forward and reverse sequencing primers in a third container, and (4) a forward sequencing capture substrate in a fourth container;
loading a nucleic acid sample including target nucleic acid in the first container and allowing the nucleic acid sample to mix with the reagent for PCR, magnetic particle, forward primer, and reverse primer in the first container;
automatically subjecting the target nucleic acid to an amplification reaction using thermal cycling to produce an amplified sample comprising hybridized forward and reverse amplification strands attached to the magnetic particle;
automatically inserting a magnet into the first container to attract the magnetic particle to which are attached the hybridized forward and reverse amplification strands; and
automatically transferring the amplified sample comprising hybridized forward and reverse amplification strands attached to the magnetic particle from the first container to the second container with the magnet for washing in the second container.

80. The method of claim 79, wherein the magnet comprises a magnetic rod contained substantially concentrically within a non-magnetic sheath and independently moveable in an axial direction relative to the non-magnetic sheath.

81. The method of claim 79, further comprising:
automatically transferring the amplified sample comprising hybridized forward and reverse amplification strands attached to the magnetic particle from the second container to the third container with the magnet;
automatically subjecting the amplified sample to an amplification reaction in the third container using thermal cycling to generate forward and reverse sequencing ladders of the amplified sample;
automatically transferring the amplified sample comprising hybridized forward and reverse amplification strands attached to the magnetic particles from the third container to the second container with the magnet, and leaving the forward and reverse sequencing ladders of the amplified sample in the third container; and
automatically transferring the forward sequencing capture substrate from the fourth container to the third container with the magnet, and hybridizing the forward sequencing capture substrate with prey moieties present on the forward sequencing ladders of the amplified sample in the third container.

82. The method of claim 81, further comprising placing (5) a wash solution in a fifth container, and (6) a denaturing agent in a sixth container.

83. The method of claim 82, further comprising:
automatically transferring the forward sequencing capture substrate hybridized with prey moieties present on the forward sequencing ladders from the third container to the fifth container with the magnet, and washing the forward sequencing capture substrate hybridized with prey moieties present on the forward sequencing ladders using the wash solution in the fifth container;
automatically transferring the forward sequencing capture substrate hybridized with prey moieties present on the forward sequencing ladders from the fifth container to the sixth container with the magnet;
denaturing the washed forward sequencing capture substrate hybridized with prey moieties present on the forward sequencing ladders in the sixth container;
selectively eluting the forward sequencing ladders in the sixth container; and
automatically transferring the forward sequencing capture substrate from the sixth container to the fourth container using the magnet.

84. The method of claim 83, further comprising automatically subjecting the forward sequencing ladders in the sixth container to capillary electrophoresis.

85. A method for increasing nucleic acid sample preparation throughput, comprising:
placing, in an apparatus configured to manipulate fluids, (1) a reagent for PCR, a magnetic particle, a forward primer, and a reverse primer in a first container, (2) a wash solution in a second container, (3) forward and reverse sequencing primers in a third container, and (4) a reverse sequencing capture substrate in a fourth container;
loading a nucleic acid sample including target nucleic acid in the first container and allowing the nucleic acid sample to mix with the reagent for PCR, magnetic particle, forward primer, and reverse primer in the first container;
automatically subjecting the target nucleic acid to an amplification reaction using thermal cycling to produce an amplified sample comprising hybridized forward and reverse amplification strands attached to the magnetic particle;
automatically inserting a magnet into the first container to attract the magnetic particle to which are attached the hybridized forward and reverse amplification strands; and
automatically transferring the amplified sample comprising hybridized forward and reverse amplification strands attached to the magnetic particle from the first container to the second container with the magnet for washing in the second container.

86. The method of claim 85, wherein the magnet comprises a magnetic rod contained substantially concentrically within a non-magnetic sheath and independently moveable in an axial direction relative to the non-magnetic sheath.

87. The method of claim 85, further comprising:
automatically transferring the amplified sample comprising hybridized forward and reverse amplification strands attached to the magnetic particle from the second container to the third container with the magnet;
automatically subjecting the amplified sample to a sequencing reaction in the third container using thermal cycling to generate forward and reverse sequencing ladders of the amplified sample;
automatically transferring the amplified sample comprising hybridized forward and reverse amplification strands attached to the magnetic particle from the third container to the second container with the magnet, and leaving the forward and reverse sequencing ladders of the amplified sample in the third container; and
automatically transferring the reverse sequencing capture substrate from the fourth container to the third container with the magnet, and hybridizing the reverse sequencing capture substrate with the reverse sequencing ladders of the amplified sample in the third container.

88. The method of claim 87, further comprising placing (5) a wash solution in a fifth container, and (6) a denaturing agent in a sixth container.

89. The method of claim 88, further comprising:
automatically transferring the reverse sequencing capture substrate hybridized with the reverse sequencing ladders from the third container to the fifth container with the magnet, and washing the reverse sequencing capture substrate hybridized with the reverse sequencing ladders using the wash solution in the fifth container;
automatically transferring the washed reverse sequencing capture substrate hybridized with the reverse sequencing ladders from the fifth container to the sixth container with the magnet;
denaturing the reverse sequencing capture substrate hybridized with the reverse sequencing ladders in the sixth container;
selectively eluting the reverse sequencing ladders in the sixth container; and
automatically transferring the reverse sequencing capture substrate from the sixth container to the fourth container using the magnet.

90. The method of claim 89, further comprising automatically subjecting the reverse sequencing ladders in the sixth container to capillary electrophoresis.

91-151. (canceled)
